(19) United States
(12) Patent Application Publication
(10) Pub. No.: US 2003/0074679 A1
(43) Pub. Date: Apr. 17, 2003

(54) Administration of nucleic acid sequence to female animal to enhance growth in offspring

(76) Inventors: Robert J. Schwartz, Houston, TX (US); Ruxandra Draghia-Akli, Houston, TX (US); Roy G. Smith, Houston, TX (US); Douglas R. Kern, The Woodlands, TX (US); Robert H. Carpenter, Bastrop, TX (US)

Correspondence Address: T. Ling Chwang, Jackson Walker L.L.P., Suite 600, 2435 N. Central Expressway, Richardson, TX, 75080, US

(21) Appl. No.: 10/021,403

(22) Filed: Dec. 12, 2001

Related U.S. Application Data
(60) Provisional application No. 60/255,021, filed on Dec. 12, 2000.

(51) Int. Cl. A01K 67/027; C12N 15/88

(52) U.S. Cl. 800/14; 800/16; 800/19; 435/455; 435/458; 800/17

(57) ABSTRACT

Growth is improved by utilizing growth enhancement potential methodology to administer a nucleic acid sequence, such as GHRH or an analog, to a female animal, preferably through a parenteral route of administration. Piglets born from sows injected with DNA encoding GHRH are larger, and effects are demonstrated in subsequent pregnancies without additional administration(s) of the vector.

This application claims priority to U.S. Provisional Patent Application No. 60/255,021 filed Dec. 12, 2000.

### FIELD OF THE INVENTION

This invention relates generally to endocrinology, medicine and cell biology. More specifically, the invention relates to the improvement of growth and performance; the stimulation of production of growth hormone in an animal at a level greater than that associated with normal growth; and the enhancement of growth utilizing the administration of DNA encoding a growth hormone releasing hormone into a female animal. Furthermore, it relates to the application of a nucleotide sequence that enhances growth, such as growth hormone releasing hormone or an analog, regulated by a muscle-specific promoter into muscle tissue, particularly using electroporation techniques.

### BACKGROUND OF THE INVENTION

The growth hormone (GH) production pathway is composed of a series of interdependent genes whose products are required for normal growth. The GH pathway genes include: (1) ligands, such as GH and insulin-like growth factor-I (IGF-I); (2) transcription factors such as prophet of pit 1, or prop 1, and pit 1; (3) agonists and antagonists, such as growth hormone releasing hormone (GHRH) and somatostatin, respectively; and (4) receptors, such as GHRH receptor (GHRH-R) and the GH receptor (GH-R). These genes are expressed in different organs and tissues, including the hypothalamus, pituitary, liver, and bone. Effective and regulated expression of the GH pathway is essential for optimal linear growth, as well as homeostasis of carbohydrate, protein, and fat metabolism GH synthesis and secretion from the anterior pituitary is stimulated by GHRH and inhibited by somatostatin, both hypothalamic hormones. The central role of GH in controlling somatic growth in humans and other vertebrates, and the physiologically relevant pathways regulating GH secretion from the pituitary are well known. GH increases production of IGF-I, primarily in the liver, and other target organs. IGF-I and GH, in turn, feedback on the hypothalamus and pituitary to inhibit GHRH and GH release. GH has both direct and indirect actions on peripheral tissues, the indirect effects being mediated mainly by IGF-I.

There is a wide spectrum of clinical conditions, both in children and adults, in which linear growth (prepubertal patients) or body composition are compromised, and which respond to GH or GHRH therapy. In all instances the GHRH-GH-IGF-I axis is functional, but not necessarily operating at optimal sensitivity or responsiveness for a variety of possible reasons.

The principal feature of GH deficiencies in children is short stature. Similar phenotypes are produced by genetic defects at different points in the GH axis (Parks et al., 1995), as well as non-GH-deficient short stature. Non-GH-deficiencies have different etiology, such as: (1) genetic diseases, Turner syndrome (Jacobs et al., 1990; Skuse et al., 1999), hypochondroplasia (Tanaka et al., 1998; Key and Gross, 1996), and Crohn's disease (Savage et al., 1999); and (2) intrauterine growth retardation (Albanese and Stanhope, 1997; Azcona et al., 1998); and (3) chronic renal insufficiency (Sohmiya et al., 1998; Benfield and Kohaut, 1997). Cases where the GH axis is unaffected (i.e., patients have normal hormones, genes and receptors) account for more than 50% of the total cases of growth retardation. In these cases GHRH or GH therapy has been shown to be effective (Gesundheit and Alexander, 1995).

Reduced GH secretion from the anterior pituitary causes skeletal muscle mass to be lost during aging from 25 years to senescence. The GHRH-GH-IGF-I axis undergoes dramatic changes through aging and in the elderly (D'Costa et al., 1993) with decreased GH production rate and GH half-life, decreased IGF-I response to GH and GHRH stimuli leading to loss of skeletal muscle mass (sarcopenia), osteoporosis, and increase in fat and decrease in lean body mass (Bartke, 1998). Previous studies have shown that in a significant number of normal elderly persons, GH and IGFs levels in serum are significantly reduced by 70-80% of their teenage level (Corpas et al., 1993; Iranmanesh et al., 1991). It has been demonstrated that the development of sarcopenia can be offset by GH therapy. However, this remains a controversial therapy in the elderly because of its cost and frequent side effects.

The production of recombinant proteins allows a useful tool for the treatment of these conditions. Although GH replacement therapy is widely used in patients with growth deficiencies and provides satisfactory growth, and may have positive psychological effects on the children being treated (Rosenbaum and Saigal, 1996; Erling, 1999), this therapy has several disadvantages, including an impractical requirement for frequent administration of GH (Monti et al., 1997; Heptulla et al., 1997) and undesirable secondary effects (Blethen et al., 1996; Watkins, 1996; Shalet et al., 1997; Allen et al, 1997).

It is well established that extracranially secreted GHRH, as mature peptide or truncated molecules (as seen with pancreatic islet cell tumors and variously located carcinoids) are often biologically active and can even produce acromegaly (Esch et al., 1982; Thorner et al., 1984). Administration of recombinant GHRH to GH-deficient children or adult humans augments IGF-I levels, increases GH secretion proportionally to the GHRH dose, yet still invokes a response to bolus doses of GHRH (Bercu and Walker, 1997). Thus, GHRH administration represents a more physiological alternative of increasing subnormal GH and IGF-I levels (Corpas et al., 1993).

Although GHRH protein therapy entrains and stimulates normal cyclical GH secretion with virtually no side effects, the short half-life of GHRH in vivo requires frequent (one to three times a day) intravenous, subcutaneous or intranasal (requiring 300-fold higher dose) administration. Thus, as a chronic treatment, GHRH administration is not practical. However, extracranially secreted GHRH, as a processed protein species (Tyr1-40 or Tyr1-Leu44) or even as shorter truncated molecules, are biologically active (Thorner et al., 1984). Importantly, a low level of GHRH (100 pg/ml) in the blood supply stimulates GH secretion (Corpas et al., 1993) and makes GHRH an excellent candidate for gene therapeutic expression. Direct plasmid DNA gene transfer is currently the basis of many emerging gene therapy strategies and thus does not require viral genes or lipid particles (Muramatsu et al., 1998; Aihara and Miyazaki, 1998). Skeletal muscle is a preferred target tissue, because muscle fiber has a long life span and can be transduced by circular DNA plasmids that express over months or years in an immunocompetent host (Davis et al., 1993; Tripathy et al., 1996). Previous reports demonstrated that human GHRH cDNA could be delivered to muscle by an injectable myogenic expression vector in mice where it transiently stimulated GH secretion to a modest extent over a period of two weeks (Draghia-Akli et al., 1997).

Wild type GHRH has a relatively short half-life in the circulatory system, both in humans (Frohman et al., 1984) and in farm animals. After 60 minutes of incubation in plasma 95% of the GHRH(1-44)NH2 is degraded, while incubation of the shorter (1-40)OH form of the hormone, under similar conditions, shows only a 77% degradation of the peptide after 60 minutes of incubation (Frohman et al., 1989). Incorporation of cDNA coding for a particular protease-resistant GHRH analog in a gene therapy vector results in a molecule with a longer half-life in serum, increased potency, and provides greater GH release in plasmid injected animals (Draghia-Akli et al., 1999, herein incorporated by reference). Mutagenesis via amino acid replacement of protease sensitive amino acids prolongs the serum half-life of the hGHRH molecule. Furthermore, the enhancement of biological activity of GHRH is achieved by using super-active analogs which may increase its binding affinity to specific receptors (Draghia-Akli et al., 1999).

There are issued patents which address administering novel GHRH analog proteins (U.S. Pat. Nos. 5,847,066; 5,846,936; 5,792,747; 5,776,901; 5,696,089; 5,486,505; 5,137,872; 5,084,442; 5,036,045; 5,023,322; 4,839,344; 4,410,512; RE33,699) or synthetic or naturally occurring peptide fragments of GHRH (U.S. Pat. Nos. 4,833,166; 4,228,158; 4,228,156; 4,226,857; 4,224,316; 4,223,021; 4,223,020; 4,223,019) for the purpose of increasing release of growth hormone. A GHRH analog containing the following mutations has been reported (U.S. Pat. No. 5,846,936): Tyr at position 1 to His; Ala at position 2 to Val, Leu, or others; Asn at position 8 to Gln, Ser, or Thr; Gly at position 15 to Ala or Leu; Met at position 27 to Nle or Leu; and Ser at position 28 to Asn. The GHRH analog which is the subject of U.S. Patent Application Serial No. 60/145,624, herein incorporated by reference, does not contain all of the amino acid substitutions reported in U.S. Pat. No. 5,846,936 to be necessary for activity. The invention of U.S. Patent Application Serial No. 60/145,624 differs from U.S. Pat. No. 5,756,264 in two respects. First, the invention of U.S. Patent Application Serial No. 60/145,624 concerns an analog of growth hormone releasing hormone which differs from the wild type form with significant modifications which improve its function as a GH secretagogue: decreased susceptibility to proteases and increased stability, which would prolong the ability to effect a therapy, and increased biological activity, which would enhance the ability to effect a therapy. The analog of U.S. Patent Application Serial No. 60/145,624 lacks the substitution at position 8 to Gln, Ser, or Thr present in the GHRG analog of U.S. Pat. No. 5,756,264. In addition, in one aspect of the invention of U.S. Patent Application Serial No. 60/145,624, the invention utilizes a DNA encoding the GHRH analog linked to a unique synthetic promoter, termed SPc5-12 (Li et al., 1999), which contains a proximal serum response element (SRE) from skeletal α-actin, multiple MEF-2 sites, MEF-1 sites, and TEF-1 binding sites, and greatly exceeds the transcriptional potencies of natural myogenic promoters. The uniqueness of such a synthetic promoter is a significant improvement over, for instance, issued patents concerning a myogenic promoter and its use (e.g. U.S. Pat. No. 5,374,544) or systems for myogenic expression of a nucleic acid sequence (e.g. U.S. Pat. No. 5,298,422).

U.S. Pat. No. 5,061,690 is directed toward increasing both birth weight and milk production by supplying to pregnant female mammals an effective amount of hGRF or one of its analogs for 10-20 days. Application of the analogs lasts throughout the lactation period. However, multiple administrations are presented, and there is no disclosure regarding administration of the growth hormone releasing hormone (or factor) as a DNA molecule, such as with gene therapy techniques.

U.S. Pat. No. 5,134,120 and 5,292,721 similarly provide no teachings regarding administration of the growth hormone releasing hormone as a DNA form. Furthermore, these patents concern exclusively multiple administrations of recombinant protein GH in the last 2 weeks of gestation and three weeks after birth. Also, no discussion is provided regarding any non-wild type form, such as is provided in the present invention.

Administration of growth hormone (GH) to farm animals enhances lean tissue deposition and/or milk production, while increasing feed efficiency (Etherton et al., 1986; Klindt et al., 1998). Numerous studies have shown that GH markedly reduces the amount of carcass fat; and consequently the quality of products increases. However, chronic GH administration has practical and physiological limitations that potentially mitigate its usefulness and effectiveness (Chung et al., 1985; Gopinath and Etherton, 1989). Experimentally, GH-releasing hormone (GHRH) was used as a more physiological alternative. For large species such as pigs or cattle, the use of GHRH, the upstream stimulator of GH, is an alternate strategy that may increase not only growth performance and milk production, but more importantly, the efficiency of production from both practical and metabolic perspectives (Dubreuil et al., 1990; Farmer et al., 1992). However, the high cost of the recombinant peptides and the required frequency of administration currently limit the widespread use of this treatment. These major drawbacks can be obviated by using a gene therapy approach to direct the ectopic production of GHRH, provided that its production could be sustained chronically. Hypothalamic tissue-specific expression of the GHRH gene is not required for activity, as extra-cranially secreted GHRH can be biologically active (Faglia et al., 1992; Melmed, 1991). A gene therapy approach to deliver GHRH is favored by the fact that the gene, cDNA and native and several mutated molecules are well characterized in swine, cattle and many other species, and that the determination of therapeutic efficacy is straightforward and unequivocal. The skeletal musculature is a perfect candidate for the target tissue, because intramuscular injection is easily performed in an industrial setting, muscle fibers have a long life span and can be transduced by circular DNA plasmids (Bettan et al., 2000; Everett et al., 2000). Thus, there is no need for re-administration and the transgene can be expressed efficiently over months or years in an immunocompetent host (Wolff et al., 1992).

### SUMMARY OF THE INVENTION

In an embodiment of the present invention there is a method of improving or enhancing growth in an offspring from a female animal comprising the step of introducing an effective amount of a vector into cells of the female animal prior to or during gestation of said offspring, wherein the vector comprises a promoter; a nucleotide sequence; and a 3′ untranslated region, under conditions wherein the nucleotide sequence is expressed and wherein the introduction and expression of the vector results in improved or enhanced growth in the offspring. In a specific embodiment, the cells of said female animal comprise diploid cells. In another specific embodiment, the cells of said female animal comprise muscle cells. In an additional specific embodiment, the nucleic acid sequence encodes a growth hormone releasing hormone or its analog. In a further specific embodiment, the growth hormone releasing hormone is SEQ ID NO:1, SEQ ID NO:8, or its respective analog. In an additional specific embodiment, the promoter comprises a synthetic myogenic promoter. In a further specific embodiment, the 3′ untranslated region comprises a hGH 3′ untranslated region. In another specific embodiment, the vector is introduced into said cells of said female animal by electroporation, through a viral vector, in conjunction with a carrier, or by parenteral route. In an additional specific embodiment, the female animal is a human, a pet animal, a farm animal, a food animal, or a work animal. In a further specific embodiment, the female animal is a human, pig, cow, sheep, goat or chicken. In an additional specific embodiment, the vector is selected from the group consisting of a plasmid, a viral vector, a liposome, and a cationic lipid. In another specific embodiment, the vector is introduced into said female in a single administration. In an additional specific embodiment, the introduction occurs during the third trimester of gestation of the offspring. In another specific embodiment, the method further comprises the step of administering to the female a ligand for a growth hormone secretagogue receptor. In another specific embodiment, the ligand administration is oral.

In an additional embodiment of the present invention there is a method of increasing levels of growth hormone in an offspring from a female animal comprising the step of introducing an effective amount of a vector into cells of the female animal prior to or during gestation of the offspring, wherein the vector comprises a promoter; a nucleotide sequence; and a 3′ untranslated region, under conditions wherein the nucleotide sequence is expressed and wherein the introduction and expression of the vector results in an increase in the levels of growth hormone in the offspring. In a specific embodiment, the cells of the female animal comprise diploid cells. In another specific embodiment, the cells of the female animal comprise muscle cells. In an additional specific embodiment, the nucleic acid sequence encodes a growth hormone releasing hormone or its analog. In a further specific embodiment, the growth hormone releasing hormone is SEQ ID NO:1, SEQ ID NO:8, or its respective analog. In an additional specific embodiment, the promoter comprises a synthetic myogenic promoter. In a further specific embodiment, the 3′ untranslated region comprises a hGH 3′ untranslated region. In another specific embodiment, the vector is introduced into the cells of the female animal by electroporation, through a viral vector, in conjunction with a carrier, or by parenteral route. In an additional specific embodiment, the female animal is a human, a pet animal, a farm animal, a food animal, or a work animal. In a further specific embodiment, the female animal is a human, pig, cow, sheep, goat or chicken. In an additional specific embodiment, the vector is selected from the group consisting of a plasmid, a viral vector, a liposome, and a cationic lipid. In another specific embodiment, the vector is introduced into the female in a single administration. In an additional specific embodiment, the introduction occurs during the third trimester of gestation of the offspring. In another specific embodiment, the method further comprises the step of administering to the female a ligand for a growth hormone secretagogue receptor. In another specific embodiment, the ligand administration is oral.

In another embodiment of the present invention there is a method of increasing lean body mass in an offspring from a female animal comprising the step of introducing an effective amount of a vector into cells of the female animal prior to or during gestation of the offspring, wherein the vector comprises a promoter; a nucleotide sequence; and a 3′ untranslated region, under conditions wherein the nucleotide sequence is expressed and wherein the introduction and expression of the vector results in increased lean body mass in the offspring. In a specific embodiment, the cells of the female animal comprise diploid cells. In another specific embodiment, the cells of the female animal comprise muscle cells. In an additional specific embodiment, the nucleic acid sequence encodes a growth hormone releasing hormone or its analog. In a further specific embodiment, the growth hormone releasing hormone is SEQ ID NO:1, SEQ ID NO:8, or its respective analog. In an additional specific embodiment, the promoter comprises a synthetic myogenic promoter. In a further specific embodiment, the 3′ untranslated region comprises a hGH 3′ untranslated region. In another specific embodiment, the vector is introduced into the cells of the female animal by electroporation, through a viral vector, in conjunction with a carrier, or by parenteral route. In an additional specific embodiment, the female animal is a human, a pet animal, a farm animal, a food animal, or a work animal. In a further specific embodiment, the female animal is a human, pig, cow, sheep, goat or chicken. In an additional specific embodiment, the vector is selected from the group consisting of a plasmid, a viral vector, a liposome, and a cationic lipid. In another specific embodiment, the vector is introduced into the female in a single administration. In an additional specific embodiment, the introduction occurs during the third trimester of gestation of the offspring. In another specific embodiment, the method further comprises the step of administering to the female a ligand for a growth hormone secretagogue receptor. In another specific embodiment, the ligand administration is oral.

In another embodiment of the present invention there is a method of increasing levels of IGF-I in an offspring from a female animal comprising the step of introducing an effective amount of a vector into cells of the female animal prior to or during gestation of said offspring, wherein the vector comprises a promoter; a nucleotide sequence; and a 3′ untranslated region, under conditions wherein the nucleotide sequence is expressed and wherein said introduction and expression of said vector results in increased levels of IGF-I in the offspring. In a specific embodiment, the cells of the female animal comprise diploid cells. In another specific embodiment, the cells of the female animal comprise muscle cells. In an additional specific embodiment, the nucleic acid sequence encodes a growth hormone releasing hormone or its analog. In a further specific embodiment, the growth hormone releasing hormone is SEQ ID NO:1, SEQ ID NO:8, or its respective analog. In an additional specific embodiment, the promoter comprises a synthetic myogenic promoter. In a further specific embodiment, the 3′ untranslated region comprises a hGH 3′ untranslated region. In another specific embodiment, the vector is introduced into the cells of the female animal by electroporation, through a viral vector, in conjunction with a carrier, or by parenteral route. In an additional specific embodiment, the female animal is a human, a pet animal, a farm animal, a food animal, or a work animal. In a further specific embodiment, the female animal is a human, pig, cow, sheep, goat or chicken. In an additional specific embodiment, the vector is selected from the group consisting of a plasmid, a viral vector, a liposome, and a cationic lipid. In another specific embodiment, the vector is introduced into said female in a single administration. In an additional specific embodiment, the introduction occurs during the third trimester of gestation of the offspring. In another specific embodiment, the method further comprises the step of administering to the female a ligand for a growth hormone secretagogue receptor. In another specific embodiment, the ligand administration is oral.

In an additional embodiment of the present invention there is a method of increasing feed efficiency in an offspring from a female animal comprising the step of introducing an effective amount of a vector into cells of the female animal prior to or during gestation of the offspring, wherein the vector comprises a promoter; a nucleotide sequence; and a 3′ untranslated region, under conditions wherein the nucleotide sequence is expressed and wherein the introduction and expression of the vector results in increased feed efficiency in the offspring. In a specific embodiment, the cells of the female animal comprise diploid cells. In another specific embodiment, the cells of the female animal comprise muscle cells. In an additional specific embodiment, the nucleic acid sequence encodes a growth hormone releasing hormone or its analog. In a further specific embodiment, the growth hormone releasing hormone is SEQ ID NO:1, SEQ ID NO:8, or its respective analog. In an additional specific embodiment, the promoter comprises a synthetic myogenic promoter. In a further specific embodiment, the 3′ untranslated region comprises a hGH 3′ untranslated region. In another specific embodiment, the vector is introduced into the cells of the female animal by electroporation, through a viral vector, in conjunction with a carrier, or by parenteral route. In an additional specific embodiment, the female animal is a human, a pet animal, a farm animal, a food animal, or a work animal. In a further specific embodiment, the female animal is a human, pig, cow, sheep, goat or chicken. In an additional specific embodiment, the vector is selected from the group consisting of a plasmid, a viral vector, a liposome, and a cationic lipid. In another specific embodiment, the vector is introduced into the female in a single administration. In an additional specific embodiment, the introduction occurs during the third trimester of gestation of the offspring. In another specific embodiment, the method further comprises the step of administering to the female a ligand for a growth hormone secretagogue receptor. In another specific embodiment, the ligand administration is oral.

In another embodiment of the present invention there is a method of increasing the rate of growth in an offspring from a female animal comprising the step of introducing an effective amount of a vector into cells of the female animal prior to or during gestation of said offspring, wherein the vector comprises a promoter; a nucleotide sequence; and a 3′ untranslated region, under conditions wherein the nucleotide sequence is expressed and wherein the introduction and expression of the vector results in increased rate of growth in the offspring. In a specific embodiment, the cells of the female animal comprise diploid cells. In another specific embodiment, the cells of the female animal comprise muscle cells. In an additional specific embodiment, the nucleic acid sequence encodes a growth hormone releasing hormone or its analog. In a further specific embodiment, the growth hormone releasing hormone is SEQ ID NO:1, SEQ ID NO:8, or its respective analog. In an additional specific embodiment, the promoter comprises a synthetic myogenic promoter. In a further specific embodiment, the 3′ untranslated region comprises a hGH 3′ untranslated region. In another specific embodiment, the vector is introduced into the cells of said female animal by electroporation, through a viral vector, in conjunction with a carrier, or by parenteral route. In an additional specific embodiment, the female animal is a human, a pet animal, a farm animal, a food animal, or a work animal. In a further specific embodiment, the female animal is a human, pig, cow, sheep, goat or chicken. In an additional specific embodiment, the vector is selected from the group consisting of a plasmid, a viral vector, a liposome, and a cationic lipid. In another specific embodiment, the vector is introduced into said female in a single administration. In an additional specific embodiment, the introduction occurs during the third trimester of gestation of the offspring. In another specific embodiment, the method further comprises the step of administering to the female a ligand for a growth hormone secretagogue receptor. In another specific embodiment, the ligand administration is oral.

In an additional embodiment of the present invention there is a method of increasing the ratio of somatotrophs to other hormone-producing cells in a pituitary gland of an offspring from a female animal comprising the step of introducing an effective amount of a vector into cells of the female animal prior to or during gestation of the offspring, wherein the vector comprises a promoter; a nucleotide sequence; and a 3′ untranslated region, under conditions wherein the nucleotide sequence is expressed and wherein the introduction and expression of the vector results in an increased ratio of somatotrophs to other hormone-producing cells in the offspring. In a specific embodiment, the cells of the female animal comprise diploid cells. In another specific embodiment, the cells of the female animal comprise muscle cells. In an additional specific embodiment, the nucleic acid sequence encodes a growth hormone releasing hormone or its analog. In a further specific embodiment, the growth hormone releasing hormone is SEQ ID NO:1, SEQ ID NO:8, or its respective analog. In an additional specific embodiment, the promoter comprises a synthetic myogenic promoter. In a further specific embodiment, the 3 untranslated region comprises a hGH 3′ untranslated region. In another specific embodiment, the vector is introduced into the cells of said female animal by electroporation, through a viral vector, in conjunction with a carrier, or by parenteral route. In an additional specific embodiment, the female animal is a human, a pet animal, a farm animal, a food animal, or a work animal. In a further specific embodiment, the female animal is a human, pig, cow, sheep, goat or chicken. In an additional specific embodiment, the vector is selected from the group consisting of a plasmid, a viral vector, a liposome, and a cationic lipid. In another specific embodiment, the vector is introduced into the female in a single administration. In an additional specific embodiment, the introduction occurs during the third trimester of gestation of the offspring. In another specific embodiment, the method further comprises the step of administering to the female a ligand for a growth hormone secretagogue receptor. In another specific embodiment, the ligand administration is oral. In a specific embodiment, the hormone-producing cells are selected from the group consisting of corticotrophs, lactotrophs and gonadotrophs.

In an additional embodiment of the present invention there is a method for delaying birth of an offspring from a female animal comprising the step of introducing an effective amount of a vector into cells of the female animal prior to or during gestation of the offspring, wherein the vector comprises a promoter; a nucleotide sequence; and a 3′ untranslated region, under conditions wherein the nucleotide sequence is expressed and wherein the introduction and expression of the vector results in delayed birth of the offspring. In a specific embodiment, the cells of the female animal comprise diploid cells. In another specific embodiment, the cells of the female animal comprise muscle cells. In an additional specific embodiment, the nucleic acid sequence encodes a growth hormone releasing hormone or its analog. In a further specific embodiment, the growth hormone releasing hormone is SEQ ID NO:1, SEQ ID NO:8, or its respective analog. In an additional specific embodiment, the promoter comprises a synthetic myogenic promoter. In a further specific embodiment, the 3′ untranslated region comprises a hGH 3′ untranslated region. In another specific embodiment, the vector is introduced into the cells of said female animal by electroporation, through a viral vector, in conjunction with a carrier, or by parenteral route. In an additional specific embodiment, the female animal is a human, a pet animal, a farm animal, a food animal, or a work animal. In a further specific embodiment, the female animal is a human, pig, cow, sheep, goat or chicken. In an additional specific embodiment, the vector is selected from the group consisting of a plasmid, a viral vector, a liposome, and a cationic lipid. In another specific embodiment, the vector is introduced into the female in a single administration. In an additional specific embodiment, the introduction occurs during the third trimester of gestation of the offspring. In another specific embodiment, the method further comprises the step of administering to the female a ligand for a growth hormone secretagogue receptor. In another specific embodiment, the ligand administration is oral.

In an additional embodiment of the present invention, there is a method of increasing milk production in an animal comprising the step of introducing an effective amount of a vector into cells of said animal, wherein said vector comprises a promoter; a nucleotide sequence; and a 3′ untranslated region linked, under conditions wherein the nucleotide sequence is expressed and wherein said introduction and expression of said vector results in increased milk production in the animal. In a specific embodiment, the cells of the female animal comprise diploid cells. In another specific embodiment, the cells of the female animal comprise muscle cells. In an additional specific embodiment, the nucleic acid sequence encodes a growth hormone releasing hormone or its analog. In a further specific embodiment, the growth hormone releasing hormone is SEQ ID NO:1, SEQ ID NO:8, or its respective analog. In an additional specific embodiment, the promoter comprises a synthetic myogenic promoter. In a further specific embodiment, the 3′ untranslated region comprises a hGH 3′ untranslated region. In another specific embodiment, the vector is introduced into the cells of the female animal by electroporation, through a viral vector, in conjunction with a carrier, or by parenteral route. In an additional specific embodiment, the female animal is a human, a pet animal, a farm animal, a food animal, or a work animal. In a further specific embodiment, the female animal is a human, pig, cow, sheep, goat or chicken. In an additional specific embodiment, the vector is selected from the group consisting of a plasmid, a viral vector, a liposome, and a cationic lipid. In another specific embodiment, the vector is introduced into the female in a single administration. In an additional specific embodiment, the introduction occurs during the third trimester of gestation of the offspring. In another specific embodiment, the method further comprises the step of administering to the female a ligand for a growth hormone secretagogue receptor. In another specific embodiment, the ligand administration is oral.

Other and further objects, features and advantages would be apparent and eventually more readily understood by reading the following specification and by reference to the accompanying drawings forming a part thereof, or any examples of the presently preferred embodiments of the invention given for the purpose of the disclosure.

### DESCRIPTION OF THE DRAWINGS

FIGS. 1A through 1C demonstrate that GHRH super-active analogs increase GH secretagogue activity and stability. FIG. 1A is a comparison of the porcine wild type (1-40)OH amino acid sequence with the analog HV-GHRH. FIG. 1B shows the effect of the different GHRH species on pig GH release in porcine primary pituitary culture. FIG. 1C demonstrates changes in stability which occur with HV-GHRH and wild type porcine GHRH during a 6 hour incubation.

FIGS. 2A through 2E demonstrate an increase in GHRH, GH and IGF-I serum levels over two months following single injections of super-active analog GHRH myogenic expression vector. FIG. 2A depicts the constructs which contain the SPc5-12 synthetic promoter and the 3′ UTR of GH. As a model of mutated protein, HV-GHRH construct was used and compared with the porcine wild type as a positive control, and with β-galactosidase construct as a negative control. FIG. 2B illustrates relative levels of serum GHRH in pSP-GHRH injected pigs versus placebo injected control pigs. FIG. 2C demonstrates absolute levels of serum GHRH in pSP-GHRH injected pigs versus controls pigs corrected for weight/blood volume increase. FIG. 2D shows variation of GH levels in pSP-HV-GHRH injected pigs. FIG. 2E shows plasma IGF-I levels following direct intramuscular injection of pSP-GHRH constructs.

FIGS. 3A through 3C demonstrate the effect of myogenic GHRH expression vectors on pig growth. FIG. 3A shows the change in average weight in injected pigs over 2 months with pSP-GHRH or pSP-HV-GHRH. FIG. 3B shows the status of feed conversion efficiency in the pSP-GHRH injected pigs versus controls. FIG. 3C is a comparison of a pSP-HV-GHRH injected pig and a placebo injected control pig, 45 days post-injection.

FIG. 4 demonstrates the effect of injection of different amounts of pSP-HV-GHRH on 10 day-old piglets.

FIG. 5 shows the effect of injection of different amounts of pSP-HV-GHRH on IGF-I levels in 10 day-old piglets.

FIG. 6 illustrates a time course for pSP-HV-GHRH plasmid injection into piglets.

FIG. 7 illustrates a preferred embodiment of the present invention for an injectable electrode versus an alternative embodiment of exterior caliper electrodes. On the top is an illustration of external caliper electrodes having 2 square plates/1.5 cm side. On the bottom is an illustration of a 6-needle array device (solid needles) with 18-26 g needles 2 cm in length present in a 1 cm diameter array. The left illustration is a side view and the right illustration is a bottom view.

FIG. 8 demonstrates birth weight of the control and experimental piglets.

FIG. 9 illustrates piglet weight at weaning for experimentals and controls.

FIG. 10 shows weight of controls cross-fostered to injected animals compared to their littermates.

FIG. 11 demonstrates weight of piglets from GHRH-treated sows cross-fostered to control sows and compared to their littermates.

FIG. 12 illustrates an overall increase in weight over the controls (fed on controls sows).

FIG. 13 shows a comparison of the experimental and control market weights.

FIG. 14 illustrates weights of the offspring at 3 weeks, 10 weeks, and 24 weeks.

FIG. 15 shows muscle weight per body weight at three weeks of age.

FIG. 16 demonstrates pituitary weight per total weight of the offspring.

FIG. 17 shows RNA analysis of GH, GHRH, and PRL in the offspring, illustrating GHRH acts in utero as a growth factor on the pituitary.

FIG. 18 illustrates DAB staining of GH-secreting cells.

FIG. 19 demonstrates IGF-I concentration in offspring at 3 weeks, 12 weeks, and 6 months.

### DETAILED DESCRIPTION OF THE INVENTION

It will be readily apparent to one skilled in the art that various substitutions and modifications may be made in the invention disclosed herein without departing from the scope and spirit of the invention.

The term “a” or “an” as used herein in the specification may mean one or more. As used herein in the claim(s), when used in conjunction with the word “comprising”, the words “a” or “an” may mean one or more than one. As used herein “another” may mean at least a second or more.

The term “animal” as used herein refers to any species of the animal kingdom. In preferred embodiments it refers more specifically to humans, animals in their wild state, animals used as pets (birds, dogs, cats, horses), animals used for work (horses, cows, dogs) and animals which produce food (chickens, cows, fish), farm animals (pigs, horses, cows, sheep, chickens) or are themselves food (frogs, chickens, fish, crabs, lobsters, shrimp, mussels, scallops, goats, boars, cows, lambs, pigs, ostrich, emu, eel) and other animals well known to the art.

The term “effective amount” as used herein is defined as the amount of the composition required to produce an effect in a host which can be monitored using several endpoints known to those skilled in the art. In a specific embodiment, these endpoints are surrogate markers.

The term “feed conversion efficiency” as used herein is defined as the amount of food an animal eats per day versus the amount of weight gained by said animal. The terms “efficiency” or “feed efficiency” as used herein is interchangeable with “feed conversion efficiency.”

The term “growth deficiencies” as used herein is defined as any health status, medical condition or disease in which growth is less than normal. The deficiency could be the result of an aberration directly affecting a growth hormone pathway (such as the GHRH-GH-IGF-I axis), indirectly affecting a growth hormone pathway, or not affecting a growth hormone pathway at all.

The term “growth hormone” as used herein is defined as a hormone which relates to growth and acts as a chemical messenger to exert its action on a target cell.

The term “growth hormone releasing hormone” as used herein is defined as a hormone which facilitates or stimulates release of growth hormone.

The term “growth hormone releasing hormone analog” as used herein is defined as a protein which contains amino acid mutations and/or deletions in the naturally occurring form of the amino acid sequence (with no synthetic dextro or cyclic amino acids), but not naturally occurring in the GHRH molecule, yet still retains its function to enhance synthesis and secretion of growth hormone.

The term “growth hormone secretagogue receptor” (GHS-R) as used herein is defined as a receptor for a small synthetic compound which is associated, either directly or indirectly, with release of growth hormone from the pituitary gland.

The term “lean body mass” as used herein is defined as the mass of the body of an animal attributed to non-fat tissue, such as muscle.

The term “ligand for a growth hormone secretagogue receptor” as used herein is defined as any compound which acts as an agonist on a growth hormone secretagogue receptor. The ligand may be synthetic or naturally occurring. The ligand may be a peptide, protein, sugar, carbohydrate, lipid, nucleic acid or a combination thereof.

The term “myogenic” as used herein refers specifically to muscle tissue.

The term “newborn” as used herein refers to an animal immediately after birth and all subsequent stages of maturity or growth.

The term “offspring” as used herein refers to a progeny of a parent, wherein the progeny is an unborn fetus or a newborn.

The term “parenteral” as used herein refers to a mechanism for introduction of material into an animal other than through the intestinal canal. In specific embodiments, parenteral includes subcutaneous, intramuscular, intravenous, intrathecal, intraperitoneal, or others.

The term “pharmaceutically acceptable” as used herein refers to a compound wherein administration of said compound can be tolerated by a recipient mammal.

The term “secretagogue” as used herein refers to a natural of synthetic molecule that enhances synthesis and secretion of a downstream-regulated molecule (e.g. GHRH is a secretagogue for GH).

The term “somatotroph” as used herein refers to a cell which produces growth hormone.

The term “therapeutically effective amount” as used herein refers to the amount of a compound administered wherein said amount is physiologically significant. An agent is physiologically significant if its presence results in technical change in the physiology of a recipient animal. For example, in the treatment of growth deficiencies, a composition which increases growth would be therapeutically effective; in consumption diseases a composition which would decrease the rate of loss or increase the growth would be therapeutically effective.

The term “vector” as used herein refers to any vehicle which delivers a nucleic acid into a cell or organism. Examples include plasmids, viral vectors, liposomes, or cationic lipids. In a specific embodiment, liposomes and cationic lipids are adjuvant (carriers) that can be complexed with other vectors to increase the uptake of plasmid or viral vectors by a target cell. In a preferred embodiment, the vector comprises a promoter, a nucleotide sequence, preferably encoding a growth hormone releasing hormone or its analog, and a 3′ untranslated region. In another preferred embodiment, the promoter, nucleotide sequence, and 3′ untranslated region are linked operably for expression in a eukaryotic cell.

The term “wasting symptoms” as used herein is defined as symptoms and conditions associated with consumption or chronic wasting diseases.

This application is related in subject matter to U.S. Provisional Patent Application No. 60/145,624, filed Jul. 26, 1999 and the corresponding U.S. Nonprovisional patent application Ser. No. 09/624,268 filed Jul. 24, 2000, both herein incorporated by reference.

To assess growth effects of the growth hormone releasing hormone (GHRH) gene therapy myogenic vectors, pregnant sows in the last trimester of gestation were injected with 10 mg of a vector containing a wild-type (pSP-wt-GHRH) or mutated (pSP-HV-GHRH) GHRH cDNA. The injection was followed by electroporation. Non-injected/electroporated sows were used as controls. The piglets from the GHRH injected sow were bigger at birth (in average 1.65±0.06 kg HV-GHRH, p<0.00002 and 1.46±0.05 kg wt-GHRH, p<0.0014, versus controls 1.27±0.02 kg). Cross-fostering studies were performed. At weaning, piglets from injected sows were bigger than controls. Cross-foster controls suckled on injected sows were significantly bigger than their littermates. The advantage was maintained, and at 170 days after birth the offspring of the injected sows averaged 135.7 kg and 129.3 kg for the HV-GHRH and wt-GHRH respectively, while the controls weight in average 125.3 kg. Multiple biochemical measurements were performed on the piglets. Total proteins were increased in piglets from injected sows, and blood urea levels were decreased at all time points tested, both constants demonstrating an improved protein catabolism. Creatinine concentration was normal, indication of a normal kidney function. Glucose and insulin levels were normal. Thus, piglets born sows treated with a gene therapy using a plasmid DNA constructs encoding for GHRH show an increase in growth pattern over normal levels to at least 170 days after birth, and are leaner, while maintaining a normal homeostasis. This increase is equally due to increase milk production in the injected sows and modification of the hypothalamic-pituitary axis in the offspring. This proof of principal experiment demonstrate that plasmid mediated transfer could be used to enhance certain animal characteristics throughout generations, while avoiding secondary effects linked with classical protein treatments.

In an embodiment of the present invention, a nucleic acid sequence is utilized in the methods of the present invention which increases growth, enhances growth, increases feed conversion efficiency, increases lean body mass, increases IGF-I levels, increases growth rate, increases the ratio of somatotrophs to other hormone-producing cells, delays birth, or increases milk production in an offspring of a female. In specific embodiments, the nucleic acid sequence is growth hormone releasing hormone, growth hormone, IGF-I, prolactin, or analogs thereof. The female may be a mother, a female who has never been pregnant or given birth before, or a surrogate mother, such as impregnated by fetal transplantation.

A preferred embodiment of the present invention utilizes the growth hormone-releasing hormone analog having the amino acid sequence of SEQ ID NO:1 or SEQ ID NO:8 (wt GHRH). As used herein, the term “wild-type” can be the endogenous form of GHRH of any animal, or it may be a slightly modified form of the hormone, such as the porcine GHRH. A skilled artisan is aware that the endogenous GHRH has 44 amino acids, and an amide group at the end, with the correct notation for that form being (1-44)NH2-GHRH. In a specific embodiment, a form with only 40 amino acids (lacking the last 4 amino acids) is used which also does not contain an amide group, and may be referred to as (1-40)OH-GHRH. This form as used herein may also be referred to as wild-type because it does not contain internal mutations if compared to the wild-type sequence, as opposed to other forms discussed herein (such as the HV) having internal mutations introduced by site-directed mutagenesis. A skilled artisan is aware that the 1-40 form and shorter forms (for example, 1-32 or 1-29) exist naturally in humans and other mammals (even in different types of GHRH secreting tumors), and they have an activity comparable with the natural (1-44)NH2. In a preferred embodiment of the present invention a GHRH with increased stability over wild type GHRH is utilized.

In other embodiments, different species of GHRH or an analog of GHRH are within the scope of the invention. In an object of the invention the residues encoded by the DNA are not modified post-translationally, given the nature of the nucleic acid administration.

The following species are within the scope of the present invention. U.S. Pat. No. 4,223,019 discloses pentapeptides having the amino acid sequence NH₂-Y-Z-E-G-J-COOH, wherein Y is selected from a group consisting of D-lysine and D-arginine; Z and J are independently selected from a group consisting of tyrosine, tryptophan, and phenylalanine; and E and G are independently selected from a group consisting of D-tyrosine, D-tryptophan, and D-phenylalanine. U.S. Pat. No. 4,223,020 discloses tetrapeptides having the following amino acid sequence NH₂-Y-Z-E-G-COOH wherein Y and G are independently selected from a group consisting of tyrosine, tryptophan, and phenylalanine; and Z and E are independently selected from a group consisting of D-tyrosine, D-tryptophan, and D-phenylalanine. U.S. Pat. No. 4,223,021 discloses pentapeptides having the following amino acid sequence NH₂-Y-Z-E-G-J-COOH wherein Y and G are independently selected from a group consisting of tyrosine, tryptophan, and phenylalanine; Z is selected from a group consisting of glycine, alanine, valine, leucine, isoleucine, proline, hydroxyproline, serine, threonine, cysteine, and methionine; and E and J are independently selected from a group consisting of D-tyrosine, D-tryptophan, and D-phenylalanine. U.S. Pat. No. 4,224,316 discloses novel pentapeptides having the following amino acid sequence NH₂-Y-Z-E-G-J-COOH wherein Y and E are independently selected from a group consisting of D-tyrosine, D-tryptophan, and D-phenylalanine; Z and G are independently selected from a group consisting of tyrosine, tryptophan, and phenylalanine; and J is selected from a group consisting of glycine, alanine, valine, leucine, isoleucine, proline, hydroxyproline, serine, threonine, cysteine, methionine, aspartic acid, glutamic acid, asparagine, glutamine, arginine, and lysine. U.S. Pat. No. 4,226,857 discloses pentapeptides having the following amino acid sequence NH₂-Y-Z-E-G-J-COOH wherein Y and G are independently selected from a group consisting of tyrosine, trytophan, and phenylalanine; Z and J are independently selected from a group consisting of D-tyrosine, D-tryptophan, and D-phenylalanine; and E is selected from a group consisting of glycine, alanine, valine, leucine, isoleucine, proline, hydroxyproline, serine, threonine, cysteine, methionine, aspartic acid, glutamic acid, asparagine, glutamine, and histidine. U.S. Pat. No. 4,228,155 discloses pentapeptides having the following amino acid sequence NH₂-Y-Z-E-G-J-COOH wherein Y is selected from a group consisting of tyrosine, D-tyrosine, tryptophan, D-tryptophan, phenylalanine, and D-phenylalanine; Z and E are independently selected from a group consisting of D-tyrosine, D-tryptophan, and D-phenylalanine; G is selected from a group consisting of lysine and arginine; and J is selected from a group consisting of glycine, alanine, valine, leucine, isoleucine, proline, hydroxyproline, serine, threonine, cysteine, and methionine. U.S. Pat. No. 4,228,156 discloses tripeptides having the following amino acid sequence NH₂-Y-Z-E-COOH wherein Y and Z are independently selected from a group consisting of D-tyrosine, D-tryptophan, and D-phenylalanine; and E is selected from a group consisting of tyrosine, tryptohan, and phenylalanine. U.S. Pat. No. 4,228,158 discloses pentapeptides having the following amino acid sequence NH₂-Y-Z-E-G-J-COOH wherein Y and G are independently selected from a group consisting of tyrosine, tryptophan, and phenylalanine, Z and E are independently selected from a group consisting of D-tyrosine, D-tryptophan, and D-phenylalanine; and J is selected from a group consisting of natural amino acids and the D-configuration thereof. U.S. Pat. No. 4,833,166 discloses a synthetic peptide having the formula: H-Asp-Pro-Val-Asn-Ile-Arg-Ala-Phe-Asp-Asp-Val-Leu-Y wherein Y is OH or NH₂ or a non-toxic salt thereof and A synthetic peptide having the formula: H-Val-Glu-Pro-Gly-Ser-Leu-Phe-Leu-Val-Pro-Leu-Pro-Leu-Leu-Pro-Val-His-Asp-Phe-Val-Gln-Gln-Phe-Ala-Gly-Ile-Y wherein Y is OH or NH₂ or a non-toxic salt thereof. Draghia-Akli et al. (1997) utilize a 228-bp fragment of hGHRH which encodes a 31-amino-acid signal peptide and an entire mature peptide human GHRH(1-44)OH (Tyr1 Leu44) originally described by Mayo et al. (1995). Guillemin et al. (1982) also determine the sequence of human pancreatic growth hormone releasing factor (hpGRF).

Additional embodiments of the present invention include: (1) a method for improving growth performance in an offspring; (2) a method for stimulating production of growth hormone in an offspring at a level greater than that associated with normal growth; and (3) a method of enhancing growth in an offspring. All of these methods include the step of introducing a plasmid vector into the mother of the offspring during gestation of the offspring or during a previous pregnancy, wherein said vector comprises a promoter; a nucleotide sequence, such as one encoding SEQ ID NO:1 or SEQ ID NO:8; and a 3′ untranslated region operatively linked sequentially at appropriate distances for functional expression.

In an additional specific embodiment there is a method for stimulating production of growth hormone in an offspring at a level greater than that associated with normal growth, said method comprising introducing into the mother of said offspring during the gestation of said offspring an effective amount of a vector, said vector comprising a promoter; a nucleotide sequence encoding SEQ ID NO:1 or SEQ ID NO:8; and a 3′ untranslated region operatively linked sequentially at appropriate distances for functional expression. A level greater than that associated with normal growth includes the basal, inherent growth of an animal with a growth-related deficiency or of an animal with growth levels similar to other similar animals in the population, including those with no growth-related deficiency.

In a preferred embodiment there is a method of enhancing growth in an animal comprising introducing into said animal an effective amount of a vector, said vector comprising a promoter; a nucleotide sequence encoding SEQ ID NO:1 or SEQ ID NO:8; and a 3′ untranslated region operatively linked sequentially at appropriate distances for functional expression. The animal whose growth is enhanced may or may not have a growth deficiency.

It is an object of the present invention to increase the growth and/or growth rate of an animal, preferably an offspring from a mother. In a preferred embodiment the growth and/or growth rate of an animal is affected for long terms, such as greater than a few weeks or greater than a few months. In a specific embodiment, this is achieved by administering growth hormone releasing hormone into the mother of the offspring, preferably in a nucleic acid form. In a preferred embodiment the GHRH nucleic acid is maintained as an episome in a muscle cell. In a specific embodiment the increase in GHRH affects the pituitary gland by increasing the number of growth hormone producing cells, and thus changes their cellular lineage. In a specific embodiment the ratio of somatotrophs (growth hormone producing cells) is increased relative to other hormone producing cells in the pituitary, such as corticotrophs, lactotrophs, gonadotrophs, etc. In a specific embodiment the increase in growth hormone, related to the increase in the number of growth hormone-producing cells, is reflected in an increase of IGF-I levels. In another specific embodiment the increase in growth hormone levels is associated with an increase in lean body mass and an increase in the rate of growth of the offspring. In another specific embodiment the increase in lean body mass is related to the increase in linear skeletal growth. In an additional specific embodiment the feed conversion efficiency of the offspring is increased. In another specific embodiment the birth of the offspring is delayed, and in a preferred embodiment this is associated with an improved or increased growth rate of the fetus.

In a preferred embodiment the promoter is a synthetic myogenic promoter and hGH 3′ untranslated region is in the 3′ untranslated region. However, the 3′ untranslated region may be from any natural or synthetic gene. In a specific embodiment of the present invention there is utilized a synthetic promoter, termed SPc5-12 (Li et al., 1999) (SEQ ID NO:6), which contains a proximal serum response element (SRE) from skeletal α-actin, multiple MEF-2 sites, MEF-1 sites, and TEF-1 binding sites, and greatly exceeds the transcriptional potencies of natural myogenic promoters. In a preferred embodiment the promoter utilized in the invention does not get shut off or reduced in activity significantly by endogenous cellular machinery or factors. Other elements, including trans-acting factor binding sites and enhancers may be used in accordance with this embodiment of the invention. In an alternative embodiment, a natural myogenic promoter is utilized, and a skilled artisan is aware how to obtain such promoter sequences from databases including the National Center for Biotechnology Information (NCBI) GenBank database or the NCBI PubMed site. A skilled artisan is aware that these World Wide Web sites may be utilized to obtain sequences or relevant literature related to the present invention.

In a specific embodiment the hGH 3′ untranslated region (SEQ ID NO:7) is utilized in a nucleic acid vector, such as a plasmid.

In specific embodiments said vector is selected from the group consisting of a plasmid, a viral vector, a liposome, or a cationic lipid. In further specific embodiments said vector is introduced into myogenic cells or muscle tissue. In a further specific embodiment said animal is a human, a pet animal, a work animal, or a food animal.

In addition to the specific embodiment of introducing said construct into the animal via a plasmid vector, delivery systems for transfection of nucleic acids into the animal or its cells known in the art may also be utilized. For example, other non-viral or viral methods may be utilized. A skilled artisan recognizes that a targeted system for non-viral forms of DNA or RNA requires four components: 1) the DNA or RNA of interest; 2) a moiety that recognizes and binds to a cell surface receptor or antigen; 3) a DNA binding moiety; and 4) a lytic moiety that enables the transport of the complex from the cell surface to the cytoplasm. Further, liposomes and cationic lipids can be used to deliver the therapeutic gene combinations to achieve the same effect. Potential viral vectors include expression vectors derived from viruses such as adenovirus, vaccinia virus, herpes virus, and bovine papilloma virus. In addition, episomal vectors may be employed. Other DNA vectors and transporter systems are known in the art.

One skilled in the art recognizes that expression vectors derived from various bacterial plasmids, retroviruses, adenovirus, herpes or from vaccinia viruses may be used for delivery of nucleotide sequences to a targeted organ, tissue or cell population. Methods which are well known to those skilled in the art can be used to construct recombinant vectors which will express the gene encoding the growth hormone releasing hormone analog. Transient expression may last for a month or more with a non-replicating vector and even longer if appropriate replication elements are a part of the vector system.

It is an object of the present invention that a single administration of a growth hormone releasing hormone is sufficient for multiple gestation periods and also provides a therapy that enhances piglets performances to the market weight, as increased growth and changed body composition.

Nucleic Acids

1. Vectors

The term “vector” is used to refer to a carrier nucleic acid molecule into which a nucleic acid sequence can be inserted for introduction into a cell where the vector can be replicated and the nucleic acid sequence can be expressed. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACS). One of skill in the art would be well equipped to construct a vector through standard recombinant techniques, which are described in Maniatis et al., 1988 and Ausubel et al., 1994, both incorporated herein by reference.

The term “expression vector” refers to a vector containing a nucleic acid sequence coding for at least part of a gene product capable of being transcribed. In a specific embodiment the nucleic acid sequence encodes part or all of GHRH. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

In a preferred embodiment, the vector of the present invention is a plasmid which comprises a synthetic myogenic (muscle-specific) promoter, a nucleotide sequence encoding a growth hormone releasing hormone or its analog, and a 3′ untranslated region. In alternative embodiments, the vectors is a viral vector, such as an adeno-associated virus, an adenovirus, or a retrovirus. In alternative embodiments, skeletal alpha-actin promoter, myosin light chain promoter, cytomegalovirus promoter, or SV40 promoter can be used. In other alternative embodiments, human growth hormone, bovine growth hormone, SV40, or skeletal alpha actin 3′ untranslated regions are utilized in the vector.

a. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one of naturally-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. In a specific embodiment the promoter is a synthetic myogenic promoter, such as is described in Li et al. (1999).

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Alnendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picomavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. No. 5,925,565 and 5,935,819, herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector.

“Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

e. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine or human growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

f. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

g. Selectable and Screenable Markers

In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

2. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5a, JM109, and KC8, as well as a number of commercially available bacterial hosts such as SURE® Competent Cells and SOLOPACKä Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC₁₂. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

3. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK® BACULOVIRUS EXPRESSION SYSTEM FROM CLONTECH®.

Other examples of expression systems include STRATAGENE®'s COMPLETE CONTROLä Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Mutagenesis

Where employed, mutagenesis will be accomplished by a variety of standard, mutagenic procedures. Mutation is the process whereby changes occur in the quantity or structure of an organism. Mutation can involve modification of the nucleotide sequence of a single gene, blocks of genes or whole chromosome. Changes in single genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

Site-Directed Mutagenesis

Structure-guided site-specific mutagenesis represents a powerful tool for the dissection and engineering of protein-ligand interactions (Wells, 1996, Braisted et al., 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multi-residue saturation mutagenesis are daunting (Warren et al., 1996, Brown et al., 1996; Zeng et al., 1996; Burton and Barbas, 1994; Yelton et al., 1995; Jackson et al., 1995; Short et al., 1995; Wong et al., 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

Dosage and Formulation

The composition (active ingredients; herein, vectors comprising a promoter; a nucleotide sequence encoding SEQ ID NO:1 or SEQ ID NO:8; and a 3′ untranslated region operatively linked sequentially at appropriate distances for functional expression) of this invention can be formulated and administered to affect a variety of growth deficiency states by any means that produces contact of the active ingredient with the agent's site of action in the body of an animal. The composition of the present invention is defined as a vector containing a nucleotide sequence encoding the compound of the invention, which is an amino acid sequence analog herein described. Said composition is administered in sufficient quantity to generate a therapeutically effective amount of said compound. One skilled in the art recognizes that the terms “administered” and “introduced” can be used interchangeably. They can be administered by any conventional means available for use in conjunction with pharmaceuticals, either as individual therapeutic active ingredients or in a combination of therapeutic active ingredients. In a preferred embodiment the active ingredient is administered alone or in a buffer such as PBS, but may be administered with a pharmaceutical carrier selected on the basis of the chosen route of administration and standard pharmaceutical practice. Such pharmaceutical compositions can be used for therapeutic or diagnostic purposes in clinical medicine, both human and veterinary. For example, they are useful in the treatment of growth-related disorders such as hypopituitary dwarfism resulting from abnormalities in growth hormone production. Furthermore they can also be used to stimulate the growth or enhance feed conversion efficiency of animals raised for meat production, to enhance milk production, and stimulate egg production.

The dosage administered will be a therapeutically effective amount of active ingredient and will, of course, vary depending upon known factors such as the pharmacodynamic characteristics of the particular active ingredient and its mode and route of administration; type of animal; age of the recipient; sex of the recipient; reproductive status of the recipient; health of the recipient; weight of the recipient; nature and extent of symptoms; kind of concurrent treatment; frequency of treatment; and the effect desired. Appropriate dosages of the vectors of the invention to be administered will vary somewhat depending on the individual subject and other parameters. The skilled practitioner will be able to determine appropriate dosages based on the known circulating levels of growth hormone associated with normal growth and the growth hormone releasing activity of the vector. As is well known in the art, treatment of a female or mother to produce bigger animals will necessitate varying dosages from individual to individual depending upon the degree of levels of increase of growth hormone production required.

Thus, there is provided in accordance with this invention a method of increasing growth of an offspring which comprises administering to the female or mother of the offspring an amount of the analog of this invention sufficient to increase the production of growth hormone to levels greater than that which is associated with normal growth. Normal levels of growth hormone vary considerably among individuals and, for any given individual, levels of circulating growth hormone vary considerably during the course of a day.

There is also provided a method of increasing the growth rate of animals by administering an amount of the inventive GHRH analog sufficient to stimulate the production of growth hormone at a level greater than that associated with normal growth.

Gene Therapy Administration

Where appropriate, the gene therapy vectors can be formulated into preparations in solid, semisolid, liquid or gaseous forms in the ways known in the art for their respective route of administration. Means known in the art can be utilized to prevent release and absorption of the composition until it reaches the target organ or to ensure timed-release of the composition. A pharmaceutically acceptable form should be employed which does not ineffectuate the compositions of the present invention. In pharmaceutical dosage forms, the compositions can be used alone or in appropriate association, as well as in combination, with other pharmaceutically active compounds.

Accordingly, the pharmaceutical composition of the present invention may be delivered via various routes and to various sites in an animal body to achieve a particular effect (see, e.g., Rosenfeld et al. (1991); Rosenfeld et al., (1991a); Jaffe et al., 1992). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

One skilled in the art recognizes that different methods of delivery may be utilized to administer a vector into a cell. Examples include: (1) methods utilizing physical means, such as electroporation (electricity), a gene gun (physical force) or applying large volumes of a liquid (pressure); and (2) methods wherein said vector is complexed to another entity, such as a liposome or transporter molecule.

Accordingly, the present invention provides a method of transferring a therapeutic gene to a host, which comprises administering the vector of the present invention, preferably as part of a composition, using any of the aforementioned routes of administration or alternative routes known to those skilled in the art and appropriate for a particular application. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

These methods described herein are by no means all-inclusive, and further methods to suit the specific application will be apparent to the ordinary skilled artisan. Moreover, the effective amount of the compositions can be further approximated through analogy to compounds known to exert the desired effect.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

The following examples are offered by way of example, and are not intended to limit the scope of the invention in any manner.

### EXAMPLE 1

### GHRH Super-Active Analogs Increase GH Secretagogue Activity and Stability

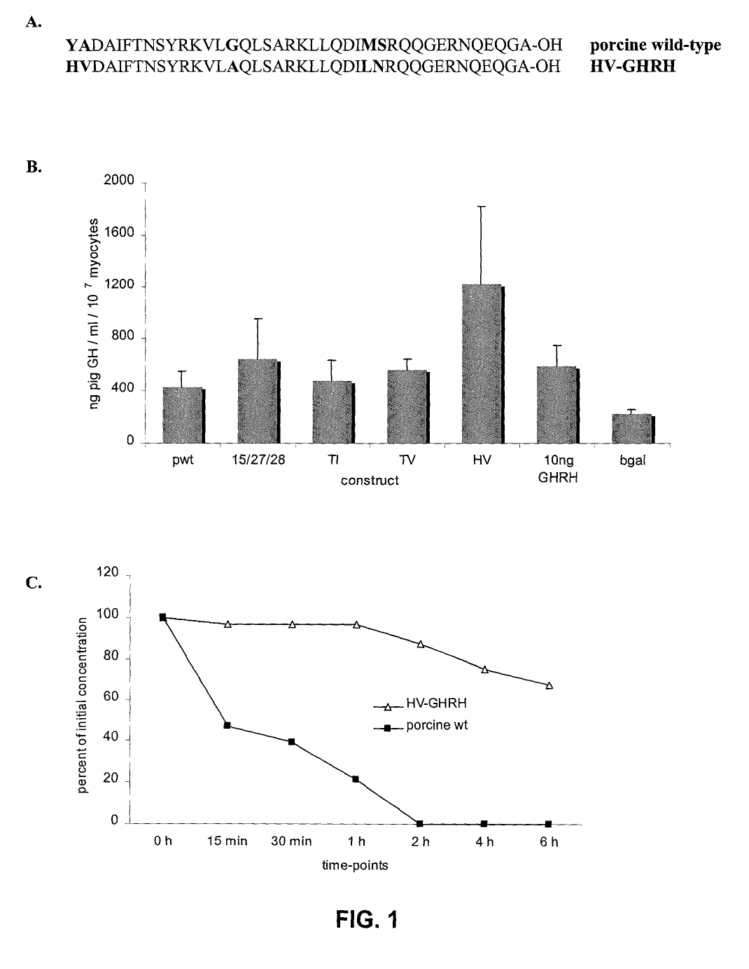

GHRH has a relatively short half-life of about 12 minutes in the circulatory systems of both humans (Frohman et al., 1984) and pigs. By employing GHRH analogs that prolong its biological half-life and/or improve its GH secretagogue activity, enhanced GH secretion is achieved. GHRH mutants were generated by site directed mutagenesis. Gly15 was substituted for Ala15 to increase α-helical conformation and amphiphilic structure to decrease cleavage by trypsin-like enzymes (Su et al., 1991). GHRH analogs with Ala15 substitutions display a 4-5 fold greater affinity for the GHRH receptor (Campbell et al., 1991). To reduce loss of biological activity due to oxidation of the Met, with slightly more stable forms using molecules with a free COOH-terminus (Kubiak et al., 1989), substitution of Met27 and Ser28 for Leu27and Asn28 was performed. Thus, a triple amino acid substitution mutant denoted as GHRH-15/27/28 was formed. Dipeptidyl peptidase IV is the prime serum GHRH degradative enzyme (Walter et al., 1980; Martin et al., 1993). Poorer dipeptidase substrates were created by taking GHRH15/27/28 and then by replacing Ile2 with Ala2 (GHRH-TI) or with Val2 (GHRH-TV), or by converting Tyr1 and Ala2 for His1 and Val2 [GHRH-HV (FIG. 1A); H1V2A15L27N28].

### EXAMPLE 2

### DNA Constructs

In a specific embodiment, a plasmid of SEQ ID NO:9 (pSPc5-12-HV-GHRH is utilized in the present invention. In another specific embodiment, a plasmid vector is utilized wherein the plasmid comprises a pVCO₂₈₉ backbone (SEQ ID NO:10); a promoter, such as of SEQ ID NO:6; a GHRH cDNA, such as the porcine HV-GHRH (the mutated HV-GHRH cDNA) (SEQ ID NO:11); and a 3′ UTR, such as from human GH (SEQ ID NO:7).

To test the biological potency of the mutated porcine GHRH cDNA sequences, plasmid vectors were engineered that were capable of directing the highest level of skeletal muscle-specific gene expression by a newly described synthetic muscle promoter, SPc5-12, which contains a proximal serum response element from skeletal α-actin, multiple MEF-2 sites, multiple MEF-1 sites, and TEF-1 binding sites (Li et al., 1999). A 228-bp fragment of porcine GHRH, which encodes the 31 amino acid signal peptide and the entire mature peptide porcine GHRH (Tyr1-Gly40) and or the GHRH mutants, followed by the 3′ untranslated region of human GH cDNA, were incorporated into myogenic GHRH expression vectors by methods well known in the art. The plasmid pSPc5-12 contains a 360 bp SacI/BamHI fragment of the SPc5-12 synthetic promoter (Li et al., 1999) in the SacI/BamHI sites of pSK-GHRH backbone (Draghia-Akli et al., 1997).

The wild type and mutated porcine GHRH cDNAs were obtained by site directed mutagenesis of human GHRH cDNA utilizing the kit Altered Sites II in vitro Mutagenesis System (Promega; Madison, Wis.). The human GHRH cDNA was subcloned as a BamHI-Hind III fragment into the corresponding sites of the pALTER Promega vector and mutagenesis was performed according to the manufacturer's directions. The porcine wild type cDNA was obtained from the human cDNA by changing the human amino acids 34 and 38 using the primer of SEQ ID NO:2: 5′-AGGCAGCAGGGAGAGAGGAACCAAGAGCAAGGAGCATAATGACTGC-AG-3′. The porcine HV mutations were made with the primer of SEQ ID NO:3: 5′-ACCCTCAGGATGCGGCGGCACGTAGATGCCATCTTCACCAAC-3′. The porcine 15Ala mutation was made with the primer of SEQ ID NO:4: 5′-CGGAAGGTGCTGGCCCAGCTGTCCGCC-3′. The porcine 27Leu28Asn mutation was made with the primer of SEQ ID NO:5: 5′-CTGCTCCAGGACATCCTGAACAGGCAGCAGGGAGAG-3′. Following mutagenesis the resulting clones were sequenced to confirm correctness and subsequently subcloned into the BamHI/Hind III sites of pSK-GHRH described in this Example by methods well known to those in the art.

### EXAMPLE 3

### Cell Culture and Transfection

Experiments were performed in both pig anterior pituitary culture and primary chicken myoblast cultures with equal success. However, the figures demonstrate data generated with pig anterior pituitary cultures. Primary chicken myoblast cultures were obtained as follows. Chicken embryonic tissue was harvested, dissected free of skin and cartilage and mechanically dissociated. The cell suspension was passed through cheesecloth and lens paper and plated at a density of 1×10⁸ to 2×10⁸/100 mm plastic culture dish. The cell populations which remained in suspension were plated at a density of 2×10⁶ to 3×10⁶ cells/collagen-coated 100 mm plastic dish and incubated at 37° C. in a 5% CO₂ environment. Cells were then incubated 24 hours prior to transfection at a density of 1.5×10⁶/100 mm plate in Minimal Essential Medium (MEM) supplemented with 10% Heat Inactivated Horse Serum (HIHS), 5% chicken embryo extract (CEE) (Gibco BRL; Grand Island, N.Y.), and gentamycin. For further details see Draghia-Akli et al., 1997 and Bergsma et al., 1986. The pig anterior pituitary culture was obtained essentially as described (Tanner et al., 1990). Briefly, pituitary tissue was dissociated under enzymatic conditions, plated on plastic dishes for enough time to allow attachment. The cells were then rinsed and exposed to incubation media prior to experiments. For details see Tanner et al. (1990).

Cells were transfected with 4 μg of plasmid per 100 mm plate, using lipofectamine, according to the manufacturer instructions. After transfection, the medium was changed to MEM which contained 2% HIHS and 2% CEE to allow the cells to differentiate. Media and cells were harvested 72 hours post-differentiation. The efficiency of transfection was estimated by β-galactosidase histochemistry of control plates to be 10%. One day before harvesting, cells were washed twice in Hank's Balanced Salt Solution (HBSS) and the media changed to MEM, 0.1% bovine serum albumin. Conditioned media was treated by adding 0.25 volume of 1% trifluoroacetic acid and 1 mM phenylmethylsulfonylflouride, frozen at −80° C., lyophilized, purified on C-18 Sep-Columns (Peninsula Laboratories, Belmont, Calif.), relyophilized and used in radioimmunoassays or resuspended in media conditioned for primary pig anterior pituitary culture.

### EXAMPLE 4

### GHRH Super-Active Analogs Increase GH Secretagogue Activity and Stability

Skeletal myoblasts were transfected as in Example 3 with each construct and GHRH moieties purified from conditioned culture media cells were assayed for growth hormone secretion in pig anterior pituitary cell cultures. As shown in FIG. 1B, media collected after 24 hours and quantitated by porcine specific GH-radioimmunoassays showed that modest gains in GH secretion amounting to about 20% to 50% for the modified GHRH species (GH15/27/28; GHRH-TI; GHRH-TV) over wild-type porcine GHRH. Only one of the four mutants, GHRH-HV, had a substantial increase in GH secretagogue activity in which porcine GH levels rose from baseline values of 200 ng/ml up to 1600 ng/ml (FIG. 1B).

### EXAMPLE 5

### Plasma Incubation of HV-GHRH Molecule

Pooled porcine plasma was collected from control pigs, and stored at -80° C. Chemically synthesized HV-GHRH was prepared by peptide synthesis. The porcine plasma was thawed and centrifuged, placed at 37° C. and allowed to equilibrate. GHRH mutant was dissolved into plasma sample to a final concentration of 100 μg/ml. Immediately after the addition of the GHRH mutant, and 15, 30, 60, 120 and 240 minutes later, 1 ml of plasma was withdrawn and acidified with 1 ml of 1M TFA. Acidified plasma was purified on C18 affinity SEP-Pak columns, lyophilized and analyzed by HPLC, using a Walters 600 multi-system delivery system, a Walters intelligent sample processor, type 717 and a Walters spectromonitor 490 (Walters Associates, Millipore Corp., Milford, Mass.). The detection was performed at 214 nm. The percent of peptide degraded at these time points was measured by integrated peak measurements.

Stability of wild type GHRH and the analog GHRH-HV was then tested in porcine plasma, by incubation of GHRH peptides, followed by solid phase extraction, and HPLC, analysis. As shown in FIG. 1C, 95% of the wild-type GHRH (1-44)NH2 was degraded within 60 minutes of incubation in plasma. In contrast, incubation of GHRH-HV in pig plasma showed that at least 75% of the polypeptides was protected against enzymatic cleavage, during 4 to 6 hours of incubation. Thus, under identical conditions, a major portion of GHRH-HV remained intact, while the wild-type GHRH is completely degraded, indicating a considerable increase in stability for GHRH-HV to serum proteases (FIG. 1C).

### EXAMPLE 6

### Animal Studies

Three groups of five, 3-4 weeks old hybrid cross barrows (Yorkshire, Landrace, Hampshire and Duroc) were used in the GHRH studies. The animals were individually housed with ad lib access to water, and 6% of their body weight diet (24% protein pig meal, Producers Cooperative Association, Bryan, Tex.). The animals were weighed every other day, at 8:30 am, and the feed was subsequently added. Animals were maintained in accordance with NIH Guide, USDA and Animal Welfare Act guidelines.

### EXAMPLE 7

### Intramuscular Injection of Plasmid DNA in Porcine

Endotoxin-free plasmid (Qiagen Inc., Chatsworth, Calif.) preparations of pSPc5-12-HV-GHRH, pSPc5-12-wt-GHRH and pSPc5-12bgal were diluted in PBS (pH 7.4) to 1 mg/ml. The animals were assigned equally to one of the treatments. The pigs were anesthetized with isoflurane (concentration of 2-6% for induction and 1-3% for maintenance). Jugular catheters were implanted by surgical procedure to draw blood from the animals at day 3, 7, 14, 21, 28, 45 and 65 post-injection. While anesthetized, 10 mg of plasmid was injected directly into the semitendinosus muscle of pigs. Two minutes after injection, the injected muscle was placed in between a set of calipers and electroporated using optimized conditions of 200V/cm with 4 pulses of 60 milliseconds (Aihara et al., 1998). At 65 days post-injection, animals were killed and internal organs and injected muscle collected, weighed, frozen in liquid nitrogen, and stored at −80° C. Carcasses were weighed and analyzed by neutron activation. Back fat was measured.

### EXAMPLE 8

### Muscle Injection of pSP-HV-GHRH Increases Porcine GHRH; GH and IGF-I Serum Levels Over 2 Months

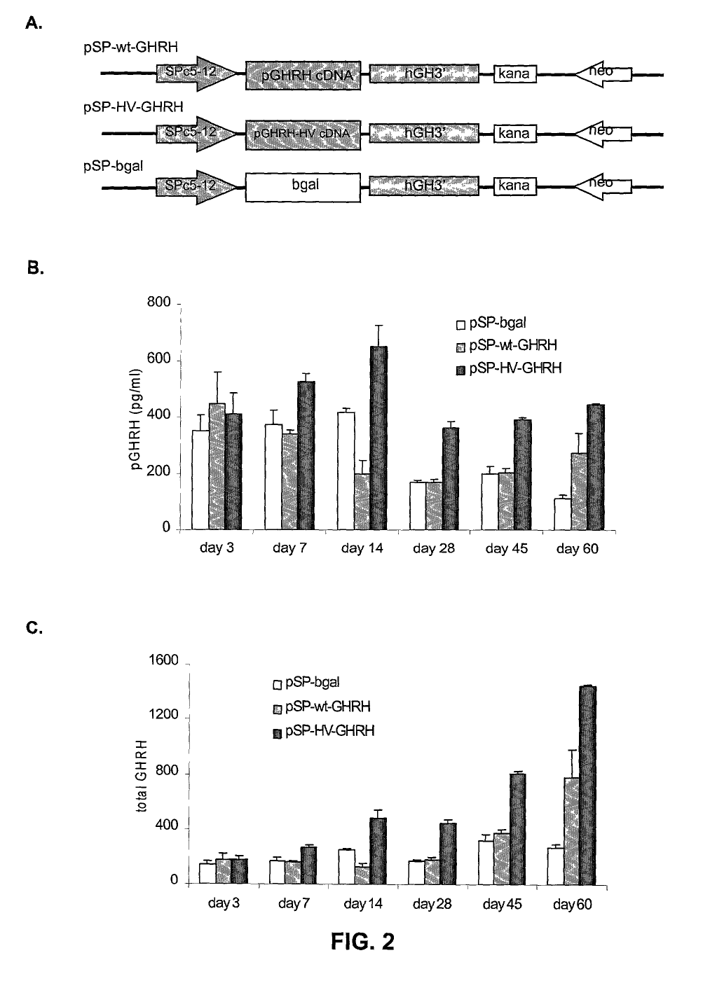

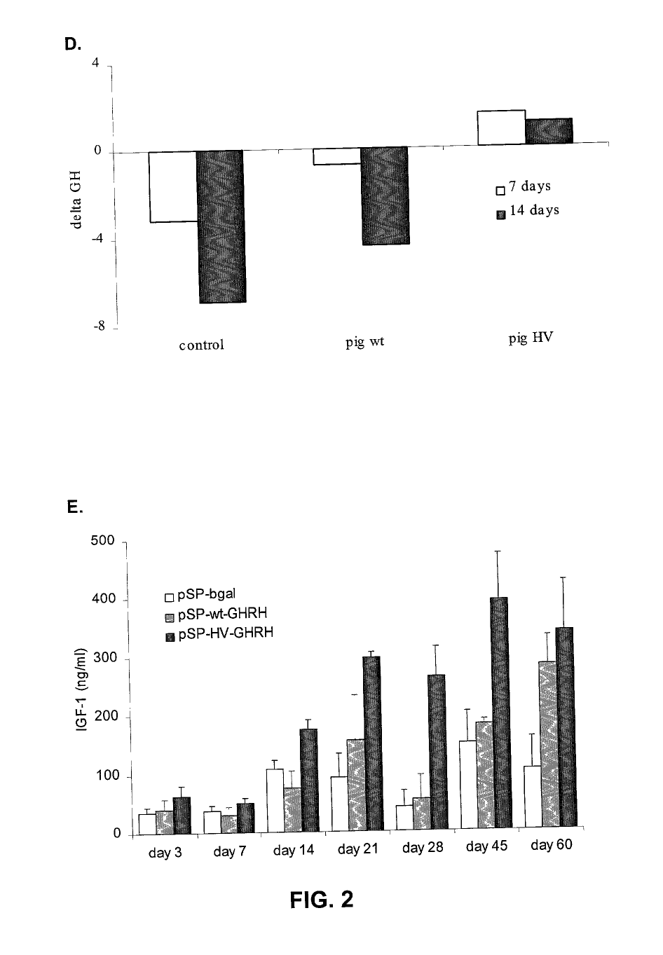

The ability of the optimized protease resistant pSP-HV-GHRH vector to facilitate long term expression of GHRH and stimulate GH and IGF-I secreted levels was determined. Schematic maps of pSP-HV-GHRH, as well as the wild-type construct, pSP-wt-GHRH, as a wild-type control, and an synthetic myogenic promoter E. coli. β-galactosidase expression vector, pSP-β gal, as the placebo control, is shown in FIG. 2A. Three-week-old castrated male-pigs were anesthetized and a jugular vein catheter was inserted to allow collection of blood samples with no discomfort for the animals. Plasmid expression vector DNA (10 mg of DNA of pSP-HV-GHRH; pSP-wt-GHRH; or pSP-β gal) was injected directly into semitendinosus muscle, which was then electroporated (See Example 7).

### EXAMPLE 9

### Porcine GHRH, GH and IGF-I Measurements

Porcine GHRH was measured by a heterologous human assay system (Peninsula Laboratories, Belmont, Calif.). Sensitivity of the assay is 1 μg/tube. Porcine GH in plasma was measured with a specific double antibody procedure RIA (The Pennsylvania State University). The sensitivity of the assay is 4 ng/tube. Porcine IGF-I was measured by heterologous human assay (Diagnostic System Lab., Webster, Tex.). Data are analyzed using Microsoft Excel statistics analysis package. Values shown in the figures are the mean ±s.e.m. Specific p values were obtained by comparison using Students t test. A p<0.05 is set as the level of statistical significance. In pigs injected in semitendinosus muscle with pSP-HV-GHRH, GHRH levels was increased at 7 days post-injection (FIG. 2B), and were 150% above the control levels at 14 days (652.4±77 pg/ml versus 419.6±13 pg/ml). pSP-HV-GHRH expression activity reached a plateau by 60 days that was about 2 to 3 fold greater levels than the placebo injected control values. The absolute quantity of serum GHRH, corrected for increased body weight between day 0 and day 60 (blood volume accounts for 8% of total body weight), secreted by the pSP-HV-GHRH injected pigs was 3 times greater than the placebo injected control values (1426.49±10.47 ng versus 266.84+25.45 ng) (FIG. 2C). The wild-type pSP-GHRH injected animals, which had been injected in semitendinosus muscle, showed only a modest increase in their GHRH levels starting with 45 days post-injection, but a 2-fold increase by 60 days post-injection (779.36 ng), at levels sufficient to elicit a biological effect.

Young animals have very high levels of GH that gradually decrease with age. Blood samples, taken every 15 minutes over a 24-hour period after the 7 and 14 days following the initial injections, were assayed for pGH levels which were extrapolated for the total change in pGH content. The pSP-HV-GHRH injected pigs (FIG. 2D) showed an increase in their GH content evident at day 7 post-injection (delta variation HV=+1.52, wt=−0.73 versus control=−3.2 ng/ml) and 14 days post-injection (delta variation HV=+1.09, wt=−4.42 versus control=−6.88 ng/ml).

Another indication of increased systemic levels of GH would be elevated levels of IGF-I. Serum porcine IGF-I levels started to rise in pSP-HV-GHRH injected pigs at about 3 days post-injection (FIG. 2E). At 21 days, these animals averaged about a 3-fold increase in serum IGF-I levels, which was maintained over 60 days (p<0.03). In comparison, pigs injected with the wild-type pSP-GHRH expression vector had only a 40% increase in their circulating IGF-I levels (p=0.39), as shown in FIG. 2E.

### EXAMPLE 10

### Myogenic GHRH Expression Vectors Enhance Pig Growth

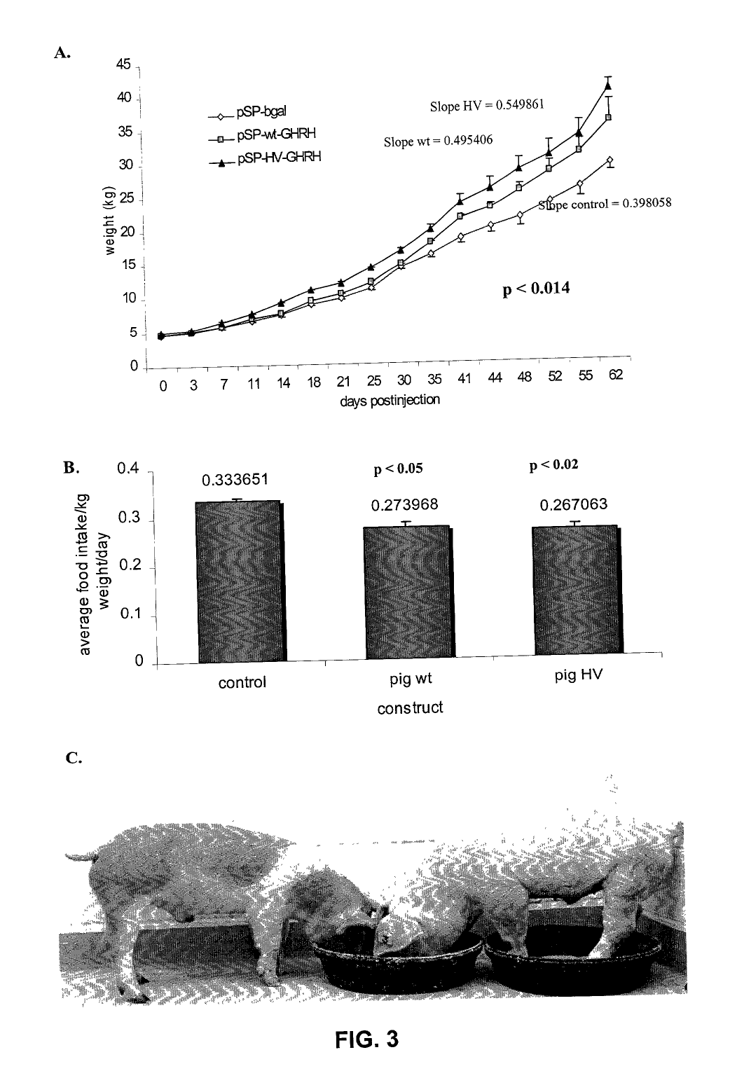

Porcine GH secreted into the systemic circulation after intramuscular injection of myogenic pSP-GHRH expression vectors augments growth over 65 days in castrated young male pigs. Body composition measurements were performed either in vivo, at day 30 and 65 post-injection (densitometry, K40) or post-mortem (organ, carcass, body fat, direct dissection followed by neutron activation chamber). Wild-type pSP-GHRH injected animals were on average 21.5% heavier than the placebo controls (37.125 kg vs. 29.375 kg), while the pSP-HV-GHRH injected pigs were 37.8% heavier (41.775 kg; p=0.014), as shown in FIG. 3A. Feed conversion efficiency was also improved by 20% in pigs injected with GHRH constructs when compared with controls (0.267 kg of food/day for each kg weight gain in pSP-HV-GHRH, and 0.274 kg in pSP-wt-GHRH, versus 0.334 kg in pSP-β gal injected pigs (FIG. 3B). Body composition studies by densitometry, K40 potassium chamber and neutron activation chamber showed a proportional increase of all body components in GHRH injected animals, with no signs of organomegaly, relative proportion of body fat and associated pathology. A photograph of a placebo injected control pig and a pSP-HV-GHRH injected pig after 45 days is shown in FIG. 3C.

The metabolic profile of pSP-HV-GHRH injected pigs shown in Table I connotes a significant decrease in serun urea level, pSP-GHRH and pSP-HV-GHRH, respectively (9±0.9 mg/dl in controls, 8.3±1 mg/dl and 6.875±0.5 mg/dl in injected pigs)(p=0.006), indicating decreased amino acid catabolism. Serum glucose level was similar between the controls and the plasmid GHRH injected pigs (99.2±4.8 mg/dl in control pigs, 104.8±6.9 mg/dl in pSP-HV-GHRH injected pigs and 97.5±8 mg/dl in wild-type pSP-GHRH injected animals (p<0.27). No other metabolic changes were found.

TABLE 1
THE METABOLIC PROFILE OF GHRH INJECTED PIGS AND
CONTROLS (VALUES IN MG/ML).

| | | | | Total |
|---|---|---|---|---|
| | Glucose | Urea | Creatinine | Protein |
| Control | 99.2 ± 4.8 | 9 ± 0.9 | 0.82 ± 0.06 | 4.6 ± 0.22 |
| pSP-wt-GHRH | 97.5 ± 8 | 8.3 ± 1 | 0.83 ± 0.056 | 4.76 ± 0.35 |
| pSP-HV-GHRH | 104.8 ± 6.9 | 6.875 ± 0.5 | 0.78 ± 0.04 | 4.88 ± 0.23 |

### EXAMPLE 11

### Experiments with Different Levels of pSP-HV-GHRH

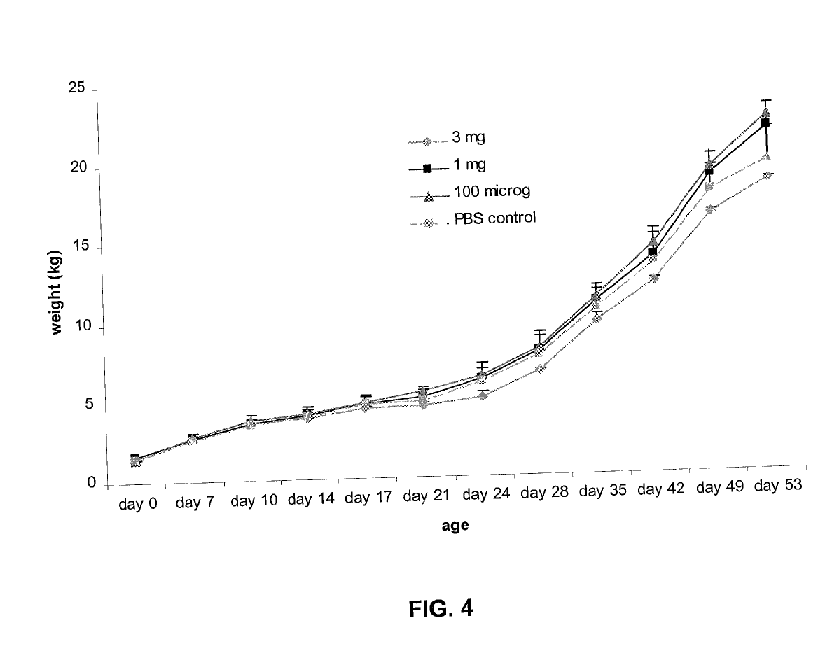

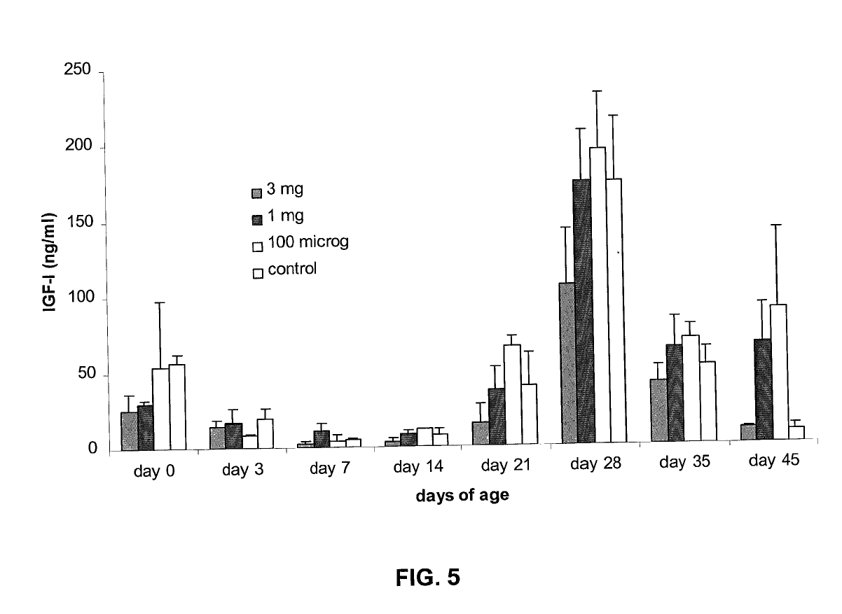

To further investigate the effects of pSP-HV-GHRH on the growth in piglets, groups of 2 piglets were injected at 10 days after birth with pSP-HV-GHRH (3 mg, 1 mg, 100 micrograms) using the new injectable six needle-array electrodes. These electrodes were previously tested and were 10-fold more efficient than caliper electrodes known in the art. Thus, needle electrodes are preferably used in methods of the present invention. As shown in FIG. 4, the group injected with 100 micrograms of the plasmid presented the best growth curve, with statistically significant differences to controls after 50 days of age. One animal in the group injected with 3 mg developed antibodies and showed a significantly decreased growth pattern.

Also, groups of 2 piglets were injected with the indicated doses of pSP-HV-GHRH 10 days after birth. IGF-I values started to rise 10 days post-injection, and at 35 days post-injection pigs injected with 100 micrograms plasmid averaged 10.62 fold higher IGF-I than the controls. Pigs injected with 1 mg averaged 7.94 fold over the controls, and pigs injected with 3 mg averaged 1.16 fold over control values.

Thus, in a specific embodiment lower dosages of pSP-HV-GHRH are injected. In a specific embodiment about 100 micrograms (0.1 milligrams) of the plasmid is utilized. In another specific embodiment about 200-300 micrograms are injected. In an additional embodiment 50-100 micrograms are administered.

### EXAMPLE 12

### Age Comparisons with pSP-HV-GHRH

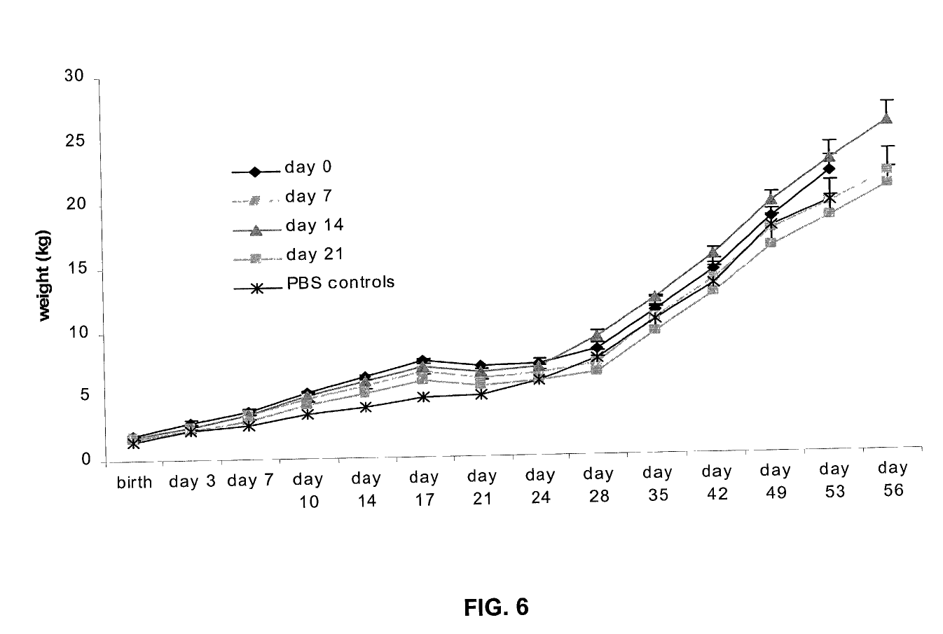

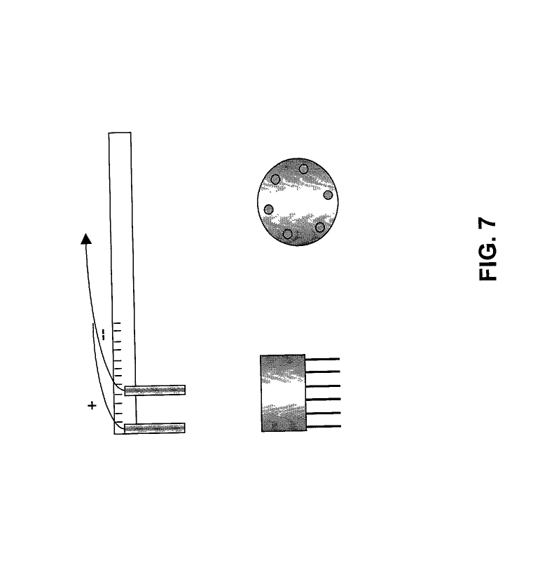

To optimize the age of piglets for pSP-HV-GHRH injection, groups of 2 piglets were injected starting at birth with 2 mg pSP-HV-GHRH. As shown in FIG. 6, the group injected 14 days after birth presented the best growth curve, with significantly statistically differences compared to the control at every time point. One animal in the group injected at 21 days developed antibodies and showed a significantly decreased growth pattern. It is possible that there is insulin resistance if treated too early (i.e., <about 10-14 days of age). In a specific embodiment the therapy is most effective when natural GH and IGF-I levels are the lowest (about 10-14 days of life), and may be counterproductive when GHRH levels are normally high. In a specific embodiment, there is a decrease in the number of antibodies produced against a modified GHRH in a pregnant animal in comparison to a non-pregnant animal, given that immune surveillance systems are reduced during pregnancy.

### EXAMPLE 13

### Specific Embodiments

In summary, an optimal time point for injection is 14 days after birth (an average 8 pounds heavier than the controls (p<0.04) at 40 days post-injection). A preferred dosage for injection is 100 micrograms plasmid in 2-5 ml volume (an average 6 pounds heavier than the controls (p<0.02) at 40 days post-injection). Hormonal and biochemical constants are normal (IGF-I, IGF-BP3, insulin, urea, glucose, total proteins, creatinine) in the offspring of sow 1 (time course) and sow 3 (dose curve) and in correlation with weight increase, with no deleterious side effects. Body composition studies from the previous experiment showed that HV-GHRH determined a uniform increase of all body compartments (body composition similar to the controls but bigger), while wt-GHRH determined an increase in lean body mass and a decrease in fat.

Given that increases in growth hormone can result in an increase in body temperature, in a preferred embodiment female pigs are injected under conditions wherein the temperature is about 62° F. to about 80° F.

### EXAMPLE 14

### Injection of the Ghrh Myogenic Vectors into Pregnant Sows Prior to the First Litter

To assay growth effects of the GHRH myogenic vectors, pregnant sows were injected with 10 mg of a vector containing a GHRH in the last trimester of gestation. In this specific example, the sow (˜800 pounds) was injected with 10 mg of a pSP-HV-GHRH vector at 90 days of gestation in her first pregnancy. Delivery methods may be any known in the art. In a specific embodiment, the plasmid is delivered as in Example 7 with the exception that a caliper electrode for electroporation was utilized (FIG. 7). The electrode has six needles 22 g which are 2 cm in length and which are on a circular plastic support of 1 cm in diameter.

Table 2 demonstrates the weight (kg) over time of piglets born from a sow injected with pSP-HV-GHRH (p2) by electroporation at 90 days of gestation. Table 3 demonstrates the weight (kg) of control animals born from an uninjected sow (p3) at the same date. Table 4 shows body composition data (fat %/BW/d mean) of the piglets from the pSP-HV-GHRH-injected sow and the uninjected sow. This table represents the relative proportion of fat to body weight and shows piglets from the injected sow had 18.5% less fat per unit of weight. Pigs p2/1 and p2/6 were sacrificed before the body composition data was obtained. Biochemistry of the piglets was similar to that demonstrated for the second pregnancy of this sow (see Example 15). The p values are very significant at all time points. These tables clearly show the piglets born from the sow injected with pSP-HV-GHRH during their gestation weigh significantly more than piglets born from the control sow. Without limiting the scope of the invention and without imposing restrictions on the metes and bounds of the invention, the Applicants surmise that the GHRH injected into muscle cells is secreted and passed through the placenta. As a result of the hypertrophic and hyperplastic effects of GHRH on the pituitary, there is an increased number of pituitary cells releasing GH.

### EXAMPLE 15

### The Second Litter of the Injected Sow

Table 5 demonstrates the weight data from the second litter of the sow injected with pSP-HV-GHRH during the first pregnancy.

TABLE 5
PIGLET BODY COMPOSITION OVER TIME

| | 27-Apr | 1-May | 5/4/2000 | 5/8/2000 | 5/11/2000 | 5/16/2000 | 5/18/200 | 5/23/2000 | 7/13/2000 |
|---|---|---|---|---|---|---|---|---|---|
| sow 2 | day 1 | day 5 | Day 7 | day 11 | Day 14 | day 19 | day 21 | day 26 | day 77 |
| pig 1 | 2.097 | 3.26 | 4.22 | 5.627 | 6.505 | 8.4 | 9.1 | 10.75 | 36.32 |
| pig 2 | 2.264 | 3.512 | 4.46 | 5.882 | 6.799 | 8.7 | 9.4 | 11.25 | 37.228 |
| pig 3 | 1.758 | 2.78 | 3.68 | 4.817 | 5.7 | 7.5 | 8.25 | 10.25 | 35.866 |
| pig 4 | 1.895 | 2.843 | 3.62 | 4.733 | 5.714 | 7.1 | 7.6 | 8.9 | 32.234 |
| pig 5 | 2.397 | 3.458 | 4.24 | 5.704 | 6.692 | 8.85 | 9.6 | 11.35 | 39.498 |
| pig 7 | 2.457 | 3.599 | 4.68 | 6.132 | 7.05 | 8.9 | 9.65 | 11.55 | 37.682 |
| pig 8 | 1.907 | 2.882 | 3.58 | 4.767 | 5.593 | 6.95 | 7.55 | 9.65 | 36.32 |
| pig 9 | 2.381 | 3.52 | 4.23 | 5.635 | 6.45 | 8.25 | 8.9 | 10.65 | 34.504 |
| pig 10 | 2.473 | 3.655 | 4.57 | 5.935 | 6.87 | 8.6 | 9.25 | 10.7 | 39.952 |
| Average | 2.181 | 3.2787 | 4.14222 | 5.47022 | 6.37478 | 8.13889 | 8.81111 | 10.56111 | 36.62267 |
| STDEV | 0.2733 | 0.3509 | 0.41817 | 0.54711 | 0.55986 | 0.75778 | 0.81616 | 0.85322 | 2.3808 |
| SE | 0.1933 | 0.2481 | 0.29569 | 0.38686 | 0.39588 | 0.53583 | 0.57711 | 0.60332 | 1.68348 |
| increase | 0 | 1.0977 | 1.96122 | 3.28922 | 4.19378 | 5.95789 | 6.63011 | 8.38011 | 34.44167 |
| sum (kg) | 19.629 | 29.509 | 37.28 | 49.232 | 57.373 | 73.25 | 79.3 | 95.05 | 329.604 |
| Pounds | 43.183 | 64.919 | 82.016 | 108.3104 | 126.2206 | 161.15 | 174.46 | 209.11 | 725.1288 |
| average | | | | | | | | 0.32231 | 0.44729 |
| daily | | | | | | | | | |
| gain | | | | | | | | | |

No subsequent administrations of GHRH were given to the sow since or during gestation with the second litter. From birth the second litter is bigger (the average for piglet weight at birth from other sows raised in a similar environment was 1.71 kg; these piglets are averaging 2.181 kg at birth). At 21 days, the sum of all the weights for the piglets in a litter characteristic for the breed and the average is ˜130pounds (˜59 kg), and the piglets from the sow previously injected with pSP-HV-GHRH are summing 174 pounds (˜79 kg). The advantage was maintained, and at 77 days after birth the weights were in average 11-15 pounds (5.5-6 kg) bigger/pig compared with the best of the breed, which are quantities well known in the art. At 169 days after birth, the injected animals were an average 22 pounds (10 kg) bigger than the controls, p<0.0007.

The sows were anesthetized only for the injection/electroporation procedure, and for them TELAZOL® (a mixture of tiletamine hydrochloride and zolazepam hydrochloride) at a dosage of 2.2 mg/kg was used. For the piglets, a combination of ketamine/xylazine HCl for the anesthesia was utilized during assessment of body composition, when the piglets must lay still on their backs in a Dual X-ray Densitometry (DEXA) machine for about 15 minutes. Specifically, ketamine 20 mg/kg+xylazine 1 mg/kg (the regular xylazine dosage is 2 mg/kg) is used. In another specific embodiment, a different anesthetic known in the art is administered, such as ketamine 15 mg/kg+acepromazine 0.4 mg/kg. In an additional specific embodiment no anesthesia in the piglets is necessary to take blood, inject, etc.

Given that pigs and some other animals are generally sensitive to different types of anesthetics and could die post-anesthesia by major changes in their thermo-regulatory process (hypo or hyperthermia, the latest much more often), atropine is sometimes administered. Atropine is an anticholinergic medication that is utilized frequently prior to anesthesia and is thought to facilitate the drying of secretions and to reduce the amount of required anesthetic, prevent cardiac arrhythmias during the procedure, and increase animal comfort during anesthetic recovery, with a decrease in the frequency of undesirable abnormal thermal episodes. In a specific embodiment there is a pretreatment with atropine at 0.05 mg/kg subq (subcutaneous). Other similar drugs known in the art may be used as an alternative to atropine.

Multiple biochemical measurements were taken of the piglets. Tables 6 through 12 provide data concerning these measurements. The insulin experiment (Table 6) was measured 5-25-00. The average of all previous control groups tested is 6.8 μU/ml, and the average of the experimental piglets is 4.785 μU/ml, with no statistical significance (p=0.07).

TABLE 6
Insulin Concentration in Piglets

| | day 25 |
|---|---|
| pig 1 | 4.3827 |
| pig 2 | 4.131 |
| pig 3 | 4.8176 |
| pig 4 | 5.7899 |
| pig 5 | 4.4267 |
| pig 7 | 4.3076 |
| pig 8 | 4.1648 |
| pig 9 | 6.0921 |
| pig 10 | 4.9527 |
| Average | 4.78501 |
| STDEV | 0.71397 |
| SE | 0.23799 |

The IGF-I assay was performed on 5-25-00 (see Table 7). The average of the experimental group is 145.509 ng/ml and the average of all previous control groups tested is 53.08 ng/ml. Therefore, the p value is very significant (p<0.0001). Given that GH stimulates production and release of IGF-I, the IGF-I assay is indicative of increases in GHRH levels and is commonly used in the art as such.

TABLE 7
IGF-I Concentration in Piglets

| | day 1 | day 10 | day 18 | day 25 |
|---|---|---|---|---|
| pig 1 | 290.46 | 118.63 | 185.01 | 356.02 |
| pig 2 | 265.7 | 115.62 | 117.99 | 172.28 |
| pig 3 | 109.27 | 77.389 | 200.75 | 109.99 |
| pig 4 | 94.689 | 36.746 | 93.795 | 65.113 |
| pig 5 | 155.98 | 95.946 | 138.24 | 179.3 |
| pig 7 | 171.41 | 19.463 | 213.29 | 226.43 |
| pig 8 | 178.3 | 101.55 | 98.478 | 165.88 |
| pig 9 | 104.86 | 78.872 | 84.7 | 77.214 |
| pig 10 | 262.4 | 131.36 | 206.23 | 138.99 |
| average | 181.4521 | 86.17511 | 148.7203 | 165.6908 |
| STDEV | 74.91415 | 37.61337 | 52.67175 | 87.96496 |
| SE | 24.97138 | 12.53779 | 17.55725 | 29.32165 |

For Table 8, the IGF-BP3 (IGF-binding protein 3) Immunoradiometric Assay (IRMA) was tested on 5-25-00. IRMA employs a two-site immunoradiometric assay (see Miles LEM, Lipschitz D A, Bieber C P and Cook J D: Measurement of serum ferritin by a 2-site immunoradiometric assay. Analyt Biochem 61:209-224, 1974). The IRMA is a non-competitive assay in which the analyte to be measured is “sandwiched” between two antibodies. The first antibody is immobilized to the inside walls of the tubes. The other antibody is radiolabelled for detection. The analyte present in the unknowns, Standards and Controls is bound by both of the antibodies to form a “sandwich” complex. Unbound materials are removed by decanting and washing the tubes. The measurements in Table 8 comprise correction factor x 50. Table 8 demonstrates the average of the experimental group is 238.88, whereas the average of all previous control groups tested is 205.44 ng/ml. There is statistical significance, with p<0.048.

TABLE 8
IGF-BP3 Concentration in Piglets

| | day 1 | day 10 | day 18 | day 25 | day 1 | day 10 | day 18 | day 25 |
|---|---|---|---|---|---|---|---|---|
| pig 1 | 7.9841 | 3.917 | 7.1657 | 3.5957 | 399.205 | 195.85 | 358.285 | 179.785 |
| pig 2 | 7.5463 | 3.4327 | 3.3382 | 4.4706 | 377.315 | 171.635 | 166.91 | 223.53 |
| pig 3 | 3.4187 | 4.9039 | 6.7961 | 6.3021 | 170.935 | 245.195 | 339.805 | 315.105 |
| pig 4 | 5.6354 | 4.2184 | 3.8551 | 1.9101 | 281.77 | 210.92 | 192.755 | 95.505 |
| pig 5 | 4.282 | 4.5592 | 5.2783 | 3.8224 | 214.1 | 227.96 | 263.915 | 191.12 |
| pig 7 | 3.7328 | 4.4454 | 2.9426 | 4.8232 | 186.64 | 222.27 | 147.13 | 241.16 |
| pig 8 | 5.4265 | 3.3285 | 4.1714 | 7.1258 | 271.325 | 166.425 | 208.57 | 356.29 |
| pig 9 | 3.7912 | 5.6354 | 3.9117 | 6.7643 | 189.56 | 281.77 | 195.585 | 338.215 |
| pig 10 | 4.7668 | 5.6099 | 5.24 | 3.8474 | 238.34 | 280.495 | 262 | 192.37 |
| average | 5.17598 | 4.45004 | 4.74434 | 4.74018 | 258.7989 | 222.5022 | 237.2172 | 237.0089 |
| STDEV | 1.652 | 0.83658 | 1.48489 | 1.70536 | 82.6 | 41.8289 | 74.24472 | 85.2679 |
| SE | 0.55067 | 0.27886 | 0.49496 | 0.56845 | 27.53333 | 13.94297 | 24.74824 | 28.42263 |

Table 9 demonstrates total protein concentration (g/dl). The average of the experimental group is 5.3 g/di, whereas the average of all previous control groups tested is 4.02 g/dl. There is very high statistical significance, with p<0.0001.

TABLE 9
Total Protein Concentration in Piglets

| | day 1 | day 10 | day 18 | day 25 |
|---|---|---|---|---|
| pig 1 | 5.7 | 5.9 G.H. | | 5.5 |
| pig 2 | 5.3 | 5.6 | 5.5 | 5 |
| pig 3 | 5.2 | 5.3 | 5.3 | 5.4 |
| pig 4 | 5.3 | 5.5 | 4.9 | 5.4 |
| pig 5 | 5.8 | 5.3 | 5 | 5.4 |
| pig 7 | 5.6 | 5.4 | 5.3 | 5.2 |
| pig 8 | 4.5 | 5 G.H. | | 4 |
| pig 9 | 5.3 | 5.1 | 5.3 | 5.2 |
| pig 10 | 6.3 | 5 | 5.2 | 5.5 |
| average | 5.44444 | 5.34444 | 5.21429 | 5.17778 |
| STDEV | 0.49526 | 0.29627 | 0.20354 | 0.47111 |
| SE | 0.16509 | 0.09876 | 0.06795 | 0.15704 |

Table 10 demonstrates creatinine concentrations (mg/dl). The average of the experimental group is 0.936 mg/dl, whereas the average of all previous control groups tested is 0.982 mg/dl. There is no statistical significance (p<0.34), which is indication of normal kidney function.

TABLE 10
Creatinine Concentration in Piglets

| | day 1 | day 10 | day 18 | day 25 |
|---|---|---|---|---|
| pig 1 | 0.75 | 0.96 G.H. | | 1.14 |
| pig 2 | 0.73 | 1.03 | 0.98 | 1.46 |
| pig 3 | 0.69 | 0.92 | 0.95 | 1.1 |
| pig 4 | 0.65 | 0.94 | 1.18 | 1.18 |
| pig 5 | 0.64 | 0.8 | 0.91 | 0.92 |
| pig 7 | 0.72 | 0.93 | 1.02 | 1.12 |
| pig 8 | 0.68 | 0.9 | 0.83 | 1.2 |
| pig 9 | 0.68 | 0.87 | 1 | 1.07 |
| pig 10 | 0.74 | 1.02 | 1.02 | 1.03 |
| average | 0.69778 | 0.93 | 0.98625 | 1.13556 |
| STDEV | 0.0393 | 0.07124 | 0.10113 | 0.14783 |
| SE | 0.0131 | 0.02375 | 0.03371 | 0.04928 |

Table 11 demonstrates BUN (blood urea levels) (mg/dl). The average of the experimental group is 3.88 mg/dl, whereas the average of all previous control groups tested is 8.119 mg/dl. There is remarkable statistical significance, with p<0.0012.

TABLE 11
BUN Concentration in Piglets

| | day 1 | day 10 | day 18 | day 25 |
|---|---|---|---|---|
| pig 1 | 4 | 3 | 5 | 4 |
| pig 2 | 4 | 3 | 3 | 6 |
| pig 3 | 6 | 6 | 5 | 7 |
| pig 4 | 5 | 3 | 4 | 5 |
| pig 5 | 3 | 2 | 3 | 3 |
| pig 7 | 3 | 3 | 3 | 3 |
| pig 8 | 2 | 3 | 5 | 7 |
| pig 9 | 3 | 3 | 4 | 4 |
| pig 10 | 3 | 3 | 3 | 4 |
| average | 3.66667 | 3.22222 | 3.88889 | 4.77778 |
| STDEV | 1.22474 | 1.09291 | 0.92796 | 1.56347 |
| SE | 0.40825 | 0.3643 | 0.30932 | 0.52116 |

Table 12 shows glucose concentrations (mg/dl). The average of the experimental group is 123.23 mg/dl, whereas the average of all previous control groups tested is 122.8 mg/dl. There is no statistical significant (p<0.67). The term G.H. stands for gross hemolysis; in these samples the determination of the biochemical constant was not possible.

TABLE 12
Glucose Concentration in Piglets

| | day 1 | day 10 | Day 18 | day 25 |
|---|---|---|---|---|
| pig 1 | 117 | 115 G.H. | | 115 |
| pig 2 | 112 | 137 | 130 | 119 |
| pig 3 | 133 | 138 | 143 | 115 |
| pig 4 | 125 | 127 | 132 | 90 |
| pig 5 | 115 | 123 | 133 | 120 |
| pig 7 | 114 | 120 | 123 | 115 |
| pig 8 | 126 | 123 G.H. | | 116 |
| pig 9 | 118 | 129 | 124 | 119 |
| pig 10 | 142 | 134 | 136 | 112 |
| Average | 122.4444 | 127.3333 | 131.5714 | 113.4444 |
| STDEV | 9.98888 | 7.88987 | 6.90066 | 9.15302 |
| SE | 3.32963 | 2.62996 | 2.30022 | 3.05101 |

As these tables demonstrate, the IGF, IGF-BP3 are increased (as a result of stimulation of GH axis), the urea and total proteins are decreased and increased respectively (which is a sign of improved protein catabolism), while insulin and glucose are maintained normal. The normal levels of insulin and glucose is an advantage to the present invention, because the classical GH therapies create a “diabetes” like situation, with hyperglycemia. Creatinine, which was normal in this experiment, is a parameter used to measure the renal function which can sometimes be impaired in animals under inappropriate metabolic conditions.

Thus, in a specific embodiment, piglets born from multiple subsequent pregnancies to the pregnancy in which the sow was first injected with pSP-HV-GHRH show an increase in growth over normal levels or animals born from sows non-injected with DNA encoding GHRH of any form. A pregnancy in pigs lasts for about 114 days, and allowing for time for lactation permits no more than 2 pregnancies/year.

In a specific embodiment, the administration of nucleic acid encoding GHRH into a female or mother is associated with an approximately 25-50% increase of GH-producing cells.

In an alternative embodiment a nonpregnant sow is injected prior to pregnancy.

In another alternative embodiment, instead of administration of the pSP-HV-GHRH vector of the present invention, other growth hormone releasing hormone analogs may be utilized, which are well known in the art. For example, wild type GHRH are used. The experiments are performed similarly to the teachings provided herein.

In another embodiment the pituitaries from the piglets are collected upon sacrifice and assayed for changes in the pituitary content. That is, the piglets will be killed and the pituitaries collected when they arrive at the market weight (˜100 kg). The assays include pituitary relative content of the different types of hormone secreting cells (relative proportion of cells secreting growth hormone, prolactine, follicle stimulating hormone (FSH), etc.)

### EXAMPLE 16

### Additional Experiments

In a specific embodiment, more sows, such as about 20, are injected with the same or similar treatments as provided in Examples 14 and 15. Multiple plasmid quantities are tested, such as from 100 micrograms to 10 milligrams, with groups of 5 sows utilized per treatment. The decedents are compared with the offspring of uninjected sows. In a specific embodiment these experiments are performed on a farm, so the data could be standardized to that in the literature.

### EXAMPLE 17

### Optimization Experiments

To determine optimum injection times during the first pregnancy, pregnant rats are utilized. The gestation in rats lasts about 21 days. Pregnant females are injected starting with day 5 to day 18 of gestation and their offspring are tested at different time points after birth. Specific experiments include the weight, body composition and pituitary relative content of the different types of hormone secreting cells (relative proportion of cells secreting growth hormone, prolactine, FSH, etc.).

### EXAMPLE 18

### Methods to Increase Milk Production

In an embodiment of the present invention there is a method to increase milk production (also termed lactation) comprising the step of introducing an effective amount of a vector into cells of an animal under conditions wherein a nucleotide sequence encoding a growth hormone releasing hormone is expressed and wherein said vector comprises a promoter; the nucleotide sequence encoding said growth hormone releasing hormone; and a 3′ untranslated region linked operatively for functional expression of said nucleotide sequence, and wherein said introduction and expression of said vector results in an increase in milk production of the animal. In a specific embodiment the animal is a human, cow, pig, goat or sheep.

Introduction of a vector comprising a GHRH by into an animal by methods described herein increases milk production in the animal. In a specific embodiment the animal is a female or mother or a pregnant female. In a further specific embodiment, the offspring of the female or mother grow faster in about the first two weeks due to the increase in milk production in the female or mother. As discussed herein, the increase in milk production occurs upon single injection of nucleic acid encoding a GHRH into an animal.

A skilled artisan is aware how to measure increases in milk production, such as in U.S. Pat. Nos. 5,061,690; 5,134,120; and 5,292,721 or in Peel et al J. Nutr., 1981, 111:1662.

Milk samples are expressed manually at the time of farrowing (colostrum) and on day 13 and day 20 of lactation. An intramuscular injection of 40 IU of oxytocin is administered (except for colostrum collection) and two glands per sow are milked as rapidly as possible until no more milk is given. The samples from the two glands are mixed thoroughly and aliquots deposited in two vials with a preservative agent, such as potassium dichromate. Vials are frozen until analysis. Milk fat, dry matter and protein is determined according to standard procedures in the art, such as A.O.A.C. (1980) procedures. In a specific embodiment milk lactose is analyzed by a semi-automated (model 27 industrial analyzer, Yellow Springs Instrument Co., Inc., Yellow Springs, Ohio) enzymatic procedure (operating procedure no. OP-025, Monsanto Co., St. Louis, Mo.). The milk yield of each sow is determined on days 13 and 20, in a specific embodiment, by weighing the pigs at hourly intervals before and after nursing as described by Lewis et al. (1978) and Mahan et al. (1971). Care is taken to prevent or account for urine and fecal losses during this time. In a specific embodiment the initial two nursing periods are used to acclimate the sow and litter and are not included in computation of the daily milk yield. Milk yield is calculated by multiplying by four the yield obtained during the subsequent 6 hours.

### EXAMPLE 19

### Other Embodiments

In another embodiment of the present invention, ligands for the growth hormone secretagogue receptor (GHS-R) give a similar result as delivery of a GHRH nucleic acid. A skilled artisan is aware of the many different GHS-R ligand structural types known in the art, all of which work through the GHS-R. Examples include MK-0677 from Merck (Whitehouse Station, N.J.), GHRP-6 (for review see Bowers, 1998) and ghrelin, an endogenous ligand (Kojima et al., 1999; Dieguez and Casanueva, 2000). Others include hexarelin (Europeptides), L-692,943 (Merck & Co.; Whitehouse Station, N.J.), NN₇O₃ (Novo Nordisk; Bagsvaerd, Denmark) or any compound which acts as an agonist on the GHS-R receptor, all of which are well known to a skilled artisan (see, for example, Pong et al. (1996); Howard et al. (1996); or Smith et al. (1997)).

A skilled artisan is aware that the GHS-R is upstream of GHRH and increases GHRH release from the pituitary gland. In a specific embodiment a GHS-R ligand is given orally (such as by adding to the feed or drinking water), which would amplify the effects of GHRH on causing release of GH from the pituitary gland. In this embodiment, the GHRH nucleic acid delivery of the present invention would get an added enhancement. Without limiting the scope of the present invention, the inventors propose that a likely mechanism of action is that the additional GHRH produces increases in the expression of pit-1 (a transcription factor involved in development of GH producing cells, somatotrophs, in anterior pituitary during embryogenesis). Activation of GHS-R also increases pit-1 expression. Pit-1 expression is also increased by cAMP, and GHS-R ligands increase the amount of cAMP made in response to GHRH. Therefore, it is likely that the pigs when born have increased concentration of somatotrophs. Hence, the pigs produce more GH. Therefore, in a specific embodiment, the GHRH nucleic acid delivery of the present invention is administered in combination with at least one GHS-R ligand. The GHS-R ligand is administered in a pharmaceutically acceptable composition

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

### EXAMPLE 20

### Multiple Effects on Sows and Offspring with GHRH Administration

In an object of the present invention, the ectopically-produced GHRH in a pregnant animal, for example, passes through the placenta to the offspring and enhances long term GH production in progeny, which then exhibit increased growth and changed body composition. In the same time, the injected sows produce significantly more milk.

To assess growth effects on the offspring of a GHRH myogenic vector injection into a large mammal and the effects of the GHRH delivery on lactation of sows, six pregnant sows were injected with 10 mg of plasmid DNA pSP-HV-GHRH (n=4) or pSP-wt-GHRH (n=2) at 95 days of gestation. Recently, significant progress toward the use of muscle for ectopic gene expression was achieved using the electroporation technique to enhance plasmid uptake in vivo, both in rodents and large mammals (Bettan et al., 2000; Draghia-Akli et al., 1999; Mir et al., 1999). In this case, plasmid injection was followed by electroporation using a 6-needle array electrode and conditions as described in herein and (Draghia-Akli et al., 1999). Six matched sows were used as controls. The animals gave birth within 24 hours of each other. A total of 132 piglets were analyzed in the subsequent studies.

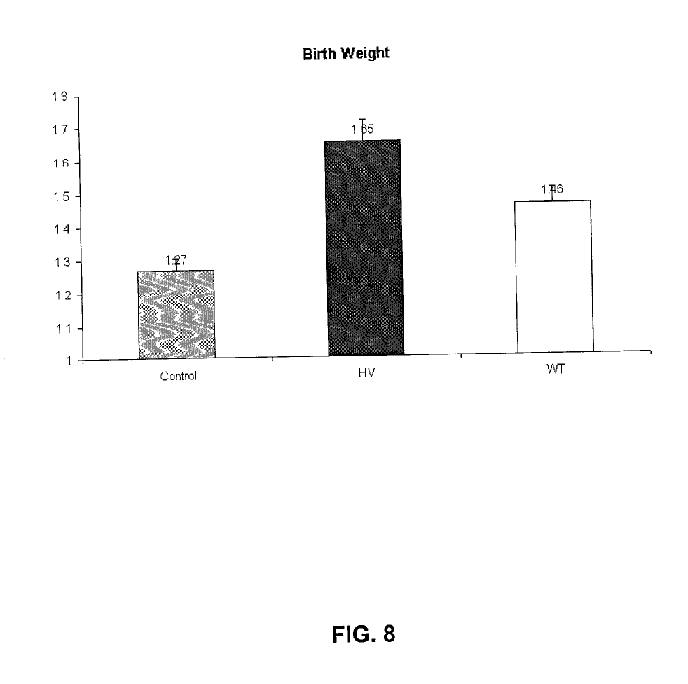

It is known that treatment with recombinant GHRH given as injections 2 weeks prior to parturition increases weight of pigs at 13 days and at weaning and improves pig survival (Etienne et al., 1992). In this case, the piglets from the GHRH injected sow were significantly bigger at birth (in average 1.65±0.06 kg HV-GHRH, p<0.00002 and 1.46±0.05 kg wt-GHRH, p<0.0014, versus controls 1.27±0.02 kg) (FIG. 8).

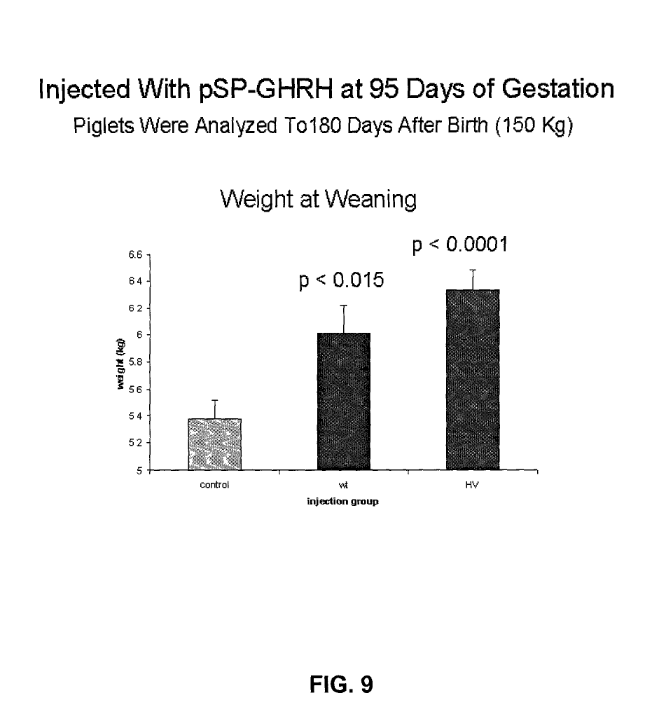

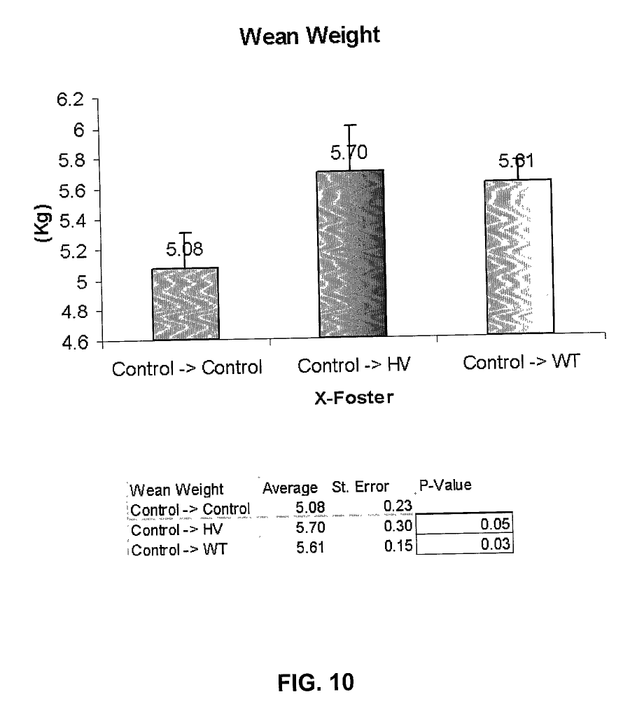

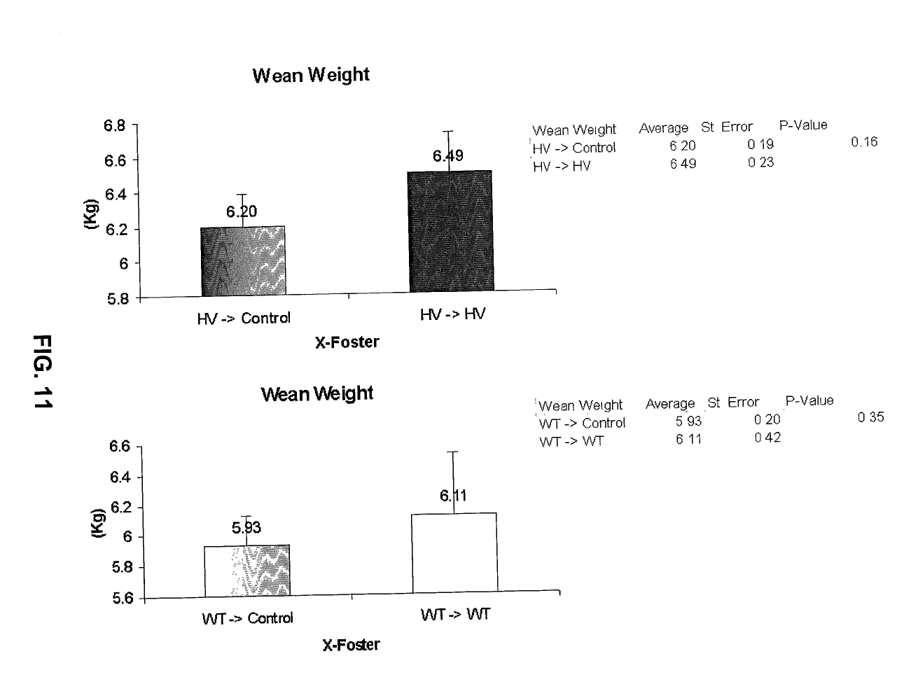

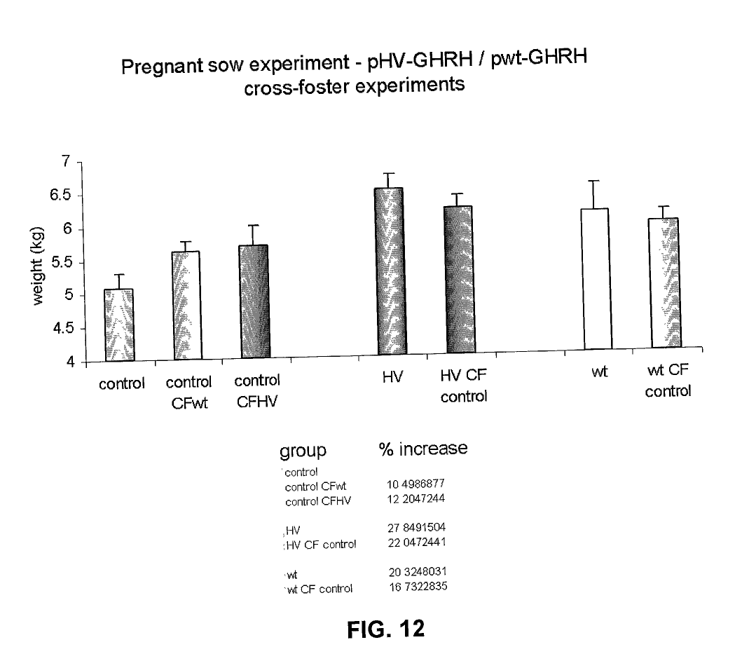

Piglets were weaned at 21 days and analyzed to slaughter weight, at 170 days after birth. Piglets from injected sows were on average 18% bigger at weaning (FIG. 9). Half of each litter was cross-fostered to either control sows (piglets from injected sows) or injected sows (piglets from control sows). Interestingly, controls cross-fostered to injected animals were significantly bigger (to up to 12.2%) than their littermates, p<0.02 (FIG. 10). This change in weight in control animals cross-fostered to GHRH treated animals is indicative of the significantly increased milk production in the injected sows. Nevertheless, piglets from GHRH-treated sows cross-fostered to control sows had a tendency to be smaller (to up to 5.8%) than their littermates (FIG. 11), but the values were not statistically significant, an indication that the offspring of GHRH treated animals have endogenous changes in their hypothalamic-pituitary axis, with increased growth. The overall increase over the controls (fed on control sows) is depicted in FIG. 12.

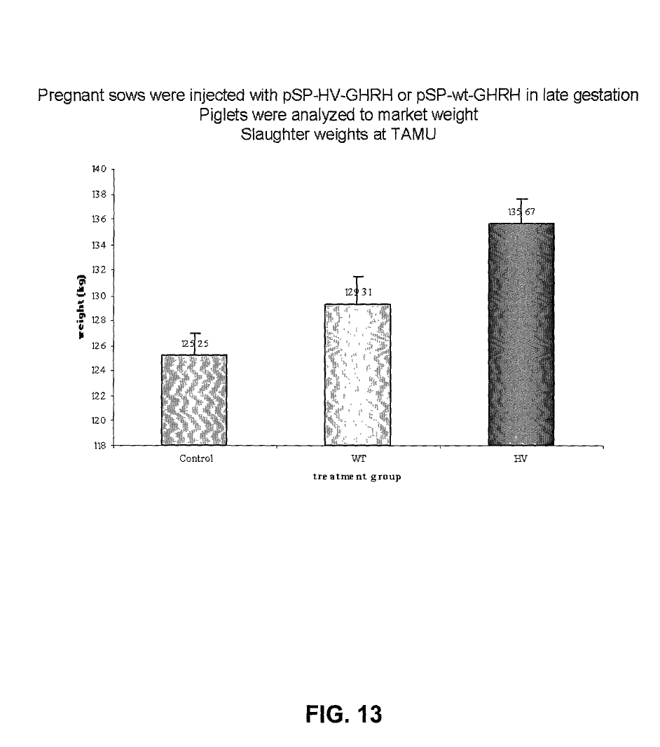

The advantage was maintained to the market weight; at 170 days the weights were on average 135.7±1.89 kg and 129.3±2.17 kg for the HV-GHRH and wt-GHRH, respectively, while the controls weight were an average of 125.3±1.74 kg (FIG. 13). The weight difference was significant statistically at every time point, with p values in between 0.05 and 10⁻⁵.

Multiple biochemical measurements were performed (Tables 13a and 13b). As a sign of increased anabolism, total protein and albumin concentration (g/dl) showed an increase in the experimental group. Total proteins increased by 8%, whereas albumin increased by 7.5%, with minor differences at the time points tested (at 50 and 170 days after birth) (Table 13a and Table 13b).

TABLE 13a

| Day 50 | Total Protein | Albumin |
|---|---|---|
| Control | 5.209 +/− .379 | 3.207 +/− .411 |
| WT-GHRH | 5.617 +/− .298 | 3.639 +/− .301 |
| p value | p < 4.3037E−05 | p < 4.83477E−05 |
| HV-GHRH | 5.533 +/− 0.291 | 3.415 +/− 0.291 |
| p value | p < 1.52284E−05 | p < 0.003470198 |

TABLE 13b

| Day 170 | Total Protein | Albumin |
|---|---|---|
| Control | 7.07 +/− 0.56 | 3.82 +/− 0.39 |
| WT | 7.68 +/− 0.31 | 4.07 +/− 0.38 |
| P-value | p < 4.045E−06 | p < 0.04199035 |
| HV | 7.33 +/− 0.29 | 4.01 +/− 0.20 |
| P-value | p < 0.00609905 | p < 0.00423639 |

Creatinine concentration (mg/dl) was normal (0.936 mg/dl versus controls 0.982 mg/di, p<0.34), which is indication of a normal kidney function.

Glucose concentrations were normal at all time points tested (Tables 14a and 14b).

TABLE 14a

| Day 50 | Glucose |
|---|---|
| Control | 99.36 +/− 12.03 |
| WT-GHRH | 98.5 +/− 10.11 |
| p value | p < 0.76483343 |
| HV-GHRH | 98.41 +/− 10.63 |
| p value | p < 0.67921581 |

TABLE 14b

| Day 170 | Insulin | Glucose |
|---|---|---|
| Control | 14.79 +/− 9.23 | 78.68 +/− 19.01 |
| WT | 10.16 +/− 2.13 | 81.14 +/− 8.90 |
| P-value | p < 0.00548803 | p < 0.49606217 |
| HV | 15.55 +/− 11.64 | 81.11 +/− 10.52 |
| P-value | p < 0.76677483 | p < 0.44978079 |

The insulin levels were normal. The normal level of insulin and glucose is an advantage because the classical GH therapies create a “diabetes”-like situation, with hyperglycemia (Pursel et al., 1990).

The survival rate over the entire study was significantly higher in offspring of the treated sows (Table 15). Morbidity was significantly reduced in the treated group.

TABLE 15

| Pig Category | Total # Pigs | # Pigs Dead | % Dead | Pathology | | Clinical Notes |
|---|---|---|---|---|---|---|
| Control | 63 | 7 | 11.11 | Sudden Death | 1 | |
| | | | | Prolapse | 1 | |
| | | | | Crippled | 1 | Rear legs |
| | | | | Enteritis | 1 | 7/26 Prolapse - |
| | | | | | | 10/10 Enteritis |
| | | | | Swollen | 2 | Tenderfooted Hermiths |
| | | | | Joints | | 8/30 Abscesses |
| | | | | Bleeding | 1 | Wasting - Anemic |
| | | | | Ulcer | | |
| WT-GHRH | 18 | 1 | 5.56 | Sudden Death | 1 | |
| HV-GHRH | 42 | 2 | 4.76 | Sudden Death | 1 | |
| | | | | Crippled | 1 | 8/21 Hurt leg fighting |

Unlike injections with porcine recombinant somatotropin (rpST) that could produce hemorrhagic ulcers, vacuolations of liver and kidney or even death of the sows (Smith et al., 1991), the GHRH gene therapy is well tolerated, and no side effects were seen in the animals. It is to be noted that the increased growth is obtained in the offspring of the treated animals, where the GHRH plasmid is not present. Regulated tissue/fibre-type-specific hGH-containing plasmids were previously used for the delivery and stable production of GH in livestock and GH-deficient hosts by either transgenesis, myoblast transfer or liposome-mediated intravenous injection (Dahler et al., 1994; Pursel et al., 1990; Barr and Leiden, 1991). Nevertheless, these techniques have significant disadvantages that preclude them from being used in a large-scale operation and/or on food animals: 1) possible toxicity or immune response associated with liposome delivery; 2) need for extensive ex vivo manipulation in the transfected myoblast approach; and/or 3) risk of important side effects or inefficiency in transgenesis (Mililer et al., 1989; Dhawan et al., 1991). Compared to these techniques, plasmid DNA injection is simple and effective, with no complication related to the delivery system or to excess expression.

The data provided herein show that enhanced biological potency is achieved in offspring of large mammals injected with a GHRH plasmid, with increased physiological levels of GH production and secretion, decreased mortality and morbidity. Treated sows display a significantly higher milk production. Offspring piglets did not experience any side effects from the therapy and had normal biochemical profiles, with no associated pathology or organomegaly. The profound enhancement in growth indicates that ectopic expression of myogenic GHRH vectors will likely replace classical GH therapy regimens and may stimulate the GH axis in a more physiologically appropriate manner. The HV-GHRH molecule, which displays a high degree of stability and GH secretory activity in pigs, may also be useful in other mammals, since the serum proteases that degrade GHRH are similar in most mammals.

The following paragraphs describe materials and methods for this Example.

DNA Constructs.

The plasmid pSPc5-12 contains a 360 bp SacI/BamHI fragment of the SPc5-12 synthetic promoter in the SacI/BamHI sites of pSK-GHRH backbone (Draghia-Akli et al., 1997). The wild type porcine GHRH was obtained by sire directed mutagenesis of human GHRH cDNA (1-40)OH at positions 34: Ser to Arg, 38: Arg to Glu; the mutated porcine HV-GHRH DNA was obtained by site directed mutagenesis of human GHRH cDNA (1-40)OH at positions 1: Tyr to His, 2 Ala to Val, 15: Gly to Ala, 27: Met to Leu, 28: Ser to Asn, 34: Ser to Arg, 38: Arg to Glu (Altered Sites II in vitro Mutagenesis System, Promega, Madison, Wis.), and cloned into the BamHI/Hind III sites of pSP-GHRH. The GHRH cDNA was followed by the 3′ untranslated region of human growth hormone, to create pSPc5-12-wt-GHRH and pSPc5-12-HV-GHRH. The control plasmid contained the E. coli beta-galactosidase gene under the control of the same synthetic promoter to create pSP-bgal.

Animal Studies.

PIC line 22 first-litter sows weighting approximate 365 kg were used in these GHRH studies. The animals were brought in the farm facility at 87 days of gestation, and individually housed in individual farrowing stalls where they remained until the end of 25 days lactation period, with ad lib access to water and food. The experiment started in March and the first litter was born in April and analyzed through mid October. The farm building was equipped with a cooling system that was able to keep the maximum temperature 2-5° C. lower that the outside temperature during hot weather. The average maximum temperatures for the month of July, August and September were 40.6° C., 41.6° C., and 36.6° C. respectively. Animals are maintained in accordance with NIH Guide, USDA and Animal Welfare Act guidelines.

Intramuscular Injection of Plasmid DNA in Porcine.

Endotoxin-free plasmid preparation of pSPc5-12-HV-GHRH and pSPc5-12-wt-GHRH (Qiagen Inc., Chatsworth, Calif., USA) were diluted in PBS pH=7.4 to 1 mg/ml. Each sow was assigned to one of treatments. Four sows were injected with pSPc5-12-HV-GHRH, two sows were injected with pSPc5-12-wt-GHRH and 6 sows were used as controls. At 95 days of gestation, animals were anesthetized lightly using telazol 2.2 mg/kg. A total of 10 mg plasmid was injected directly into the left semitendinosous muscle of pigs. Two minutes later, the injected muscle was electroporated using 6-needle array injectable electrodes, 1 cm diameter, 22 gauge, 2 cm length, using the following conditions: 6 pulses, alternate field in between needles, 200V/cm, 60 milliseconds/pulse, as described (Draghia-Akli et al., 1999; Aihara and Miyazai, 1998).

Cross-Fostering Studies.

Immediately after birth each litter was divided into two groups. A half of each litter remained on its own mother, and a half of the litter was cross-fostered to a different group (e.g. control piglets were cross-fostered to HV- or wt-injected animals, HV or wt born piglets were cross-fostered on control animals. the weight were recorded weekly.

Diet.

After weaning at 21 days, the piglets were fed for 60 days Nutrena 18% Medicated Pig Starter with 1.012% Lysine (Cargill, Minneapolis, Minn.). Subsequently, pigs were fed a Custom Mix Pig Starter 24% protein with 1.4% lysine for 45 days, Custom Mix 22.7% protein with 1.4% lysine for 45 days, and then maintained on a Custom Mix with 20% protein with 1.2% lysine (Cargill, Minneapolis, Minn.) for the rest of the study.

Biochemistry.

Serum was collected at 50 days and 170 days after birth, and analyzed by an independent laboratory (Antech Diagnostics, Irvine, Calif.).

Porcine IGF-I RIA.

Porcine IGF-I was measured by heterologous human IGF-I assay (Diagnostic System Lab., Webster, Tex.).

Porcine Insulin RIA.

Porcine insulin was measured by heterologous human assay (Linco Research Inc.; St. Charles, Mo.). The sensitivity of the assay was 2 microU/ml.

Body Composition Data.

Weights were measured on the same calibrated scales (certified to have an accuracy to ±0.2 kg and a coefficient of variation of 0.3%) throughout the study, twice a week.

Statistics.

Data are analyzed using Microsoft Excel statistics analysis package. Values shown in the figures are the mean ±s.e.m. Specific p values will be obtained by comparison using Students t test. A p<0.05 was set as the level of statistical significance.

### EXAMPLE 21

### Multiple Effects on Rats Treated with GHRH

Secretion of growth hormone (GH) is stimulated by the natural GH secretagogue, growth hormone releasing hormone (GHRH), and inhibited by stomatostatin (SS), both hypothalamic hormones (Thorner et al., 1995). GH pulses are a result of GHRH secretion that are associated with a diminution or withdrawal of somatostatin secretion. In addition, the pulse generator mechanism appears to be timed by GH-negative feedback. Additionally, ghrelin, a novel peptide initially isolated from the rat stomach, has been recognized as an important regulator of GH secretion and energy homeostasis. Ghrelin is the endogenous ligand of the growth hormone secretagogue receptor and its GH-releasing activity in vivo is dependent on GHRH (Hataya et al., 2001). In healthy adult mammals, GH is released in a highly regulated, distinctive pulsatile pattern, which occurs 4-8 times within 24 h, and has profound importance for its biological activity (Argente et al., 1996). The episodic pattern of secretion relates to the optimal induction of physiological effects at a peripheral level (Veldurs, 1998). The expression, processing, and/or release of GH isoforms and the relative proportion in between them are under differential control during growth and developmental stage (Araburo et al., 2000).

Regulation and differentiation of somatotrophs also depend upon paracrine processes within the pituitary itself and involve growth factors and several neuropeptides, for instance, vasoactive intestinal peptide (Rawlings et al., 1995), angiotensin 2, endothelin (Tomic et al., 1999), and activin (Billesbup et al., 1990). Effective and regulated expression of the GH and insulin-like growth factor I (IGF-I) pathway is essential for optimal linear growth, homeostasis of carbohydrate, protein, and fat metabolism, and for providing a positive nitrogen balance (Murray and Shalet, 2000). GHRH, GH, ghrelin, prolactin (PRL) and IGF-I play a significant role in regulation of the humoral and cellular immune responses in physiological as well as pathological situations (Geffner et al., 1997; Hattori et al., 2001).

Hypothalamic tissue-specific expression of the GHRH gene is not required for activity, as extra-cranially secreted GHRH can be biologically active (Faglia et al., 1992; Melmed, 1991). Pathological GHRH stimulation (irrespective of its source, from transgenic models to pancreatic tumors) of GH activity can result in proliferation, hyperplasia, and adenoma of adenohypophysial cells (Asa et al., 1992; Sano et al., 1988). Nevertheless, the long-term effects of a sustained GHRH treatment on the offspring of the animals receiving the therapy is yet unknown.

It has previously been shown that ectopic expression of a novel, serum protease resistant, porcine GHRH directed by an expression plasmid that was controlled by a synthetic muscle-specific promoter elicited high GH and IGF-I levels in pigs following delivery by intramuscular injection and in vivo electroporation (Lopez-Calderon et al., 1999). The purpose of the experiments described in this Example was to evaluate the GHRH delivered by plasmid DNA gene therapy to enhance growth and change body composition in the offspring of animals treated during the last trimester of gestation.

In a specific embodiment, the ectopically-produced GHRH in a pregnant animal passes through the placenta to the offspring, determines pituitary hyperplasia and enhances long term GH production in progeny, which would then exhibit increased growth and changed body composition. To assess growth effects on the offspring of a GHRH myogenic vector injection into a mammal, pregnant rats were injected with 30 μg of plasmid DNA pSP-HV-GHRH or pSP-βgal at 16 days of gestation. The injection was followed by electroporation, to enhance plasmid uptake.

All animals gave birth at 20-22 days of gestation. The average number of offspring in litters was similar in between groups (treated (T), n=10.8 pups/litter; controls (C)n=11.75 pups/litter). The number of pups was equalized in between mothers at 10 pups /mother. At two weeks after birth, the average weight in litters was 9% increased for the treated group: T=31.47±0.52 g vs. C=28.86±0.75 g, p<0.014.

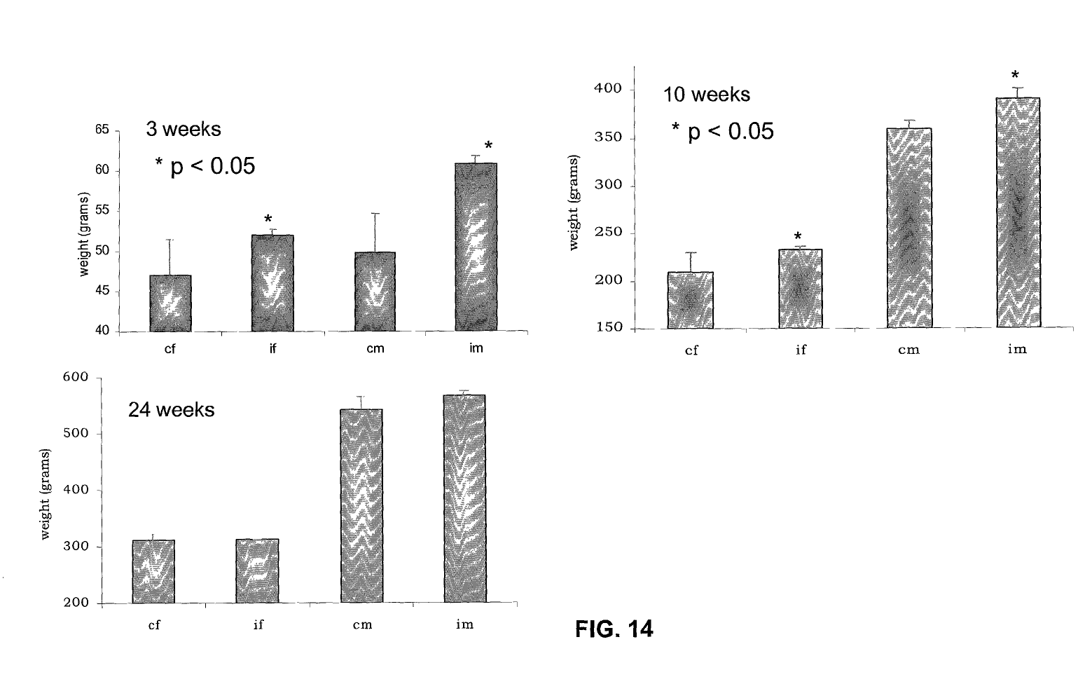

At weaning, weights were significantly increased in the offspring of T: T females (TF) averaged 51.97±0.83 g versus control females (CF) 47.07±4.4 g, p<0.043, and treated males averaged 60.89±1.02 g versus control males (CM) 49.85±4.9 g, p<0.001 (FIG. 14). The advantage was maintained to 10 weeks of age, and the weight difference became insignificant by 24 weeks.

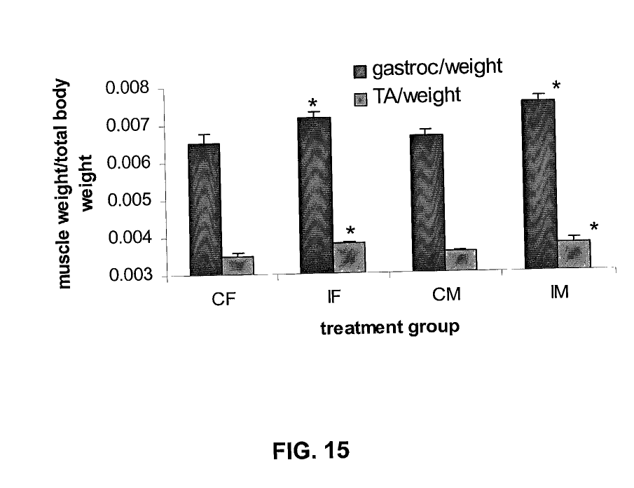

Both sexes had muscle hypertrophy at 3 weeks of age with significant differences in the gastrocnemius (G) and tibialis anterior (TA) muscles/weight (FIG. 15). TF maintained muscle hypertrophy throughout the study, while males did not show signs of muscle hypertrophy after 10 weeks of age. This change is probably attributed to changes in the sexual steroids at maturity in males that blunt the effects of physiologically increased GH on the skeletal muscle.

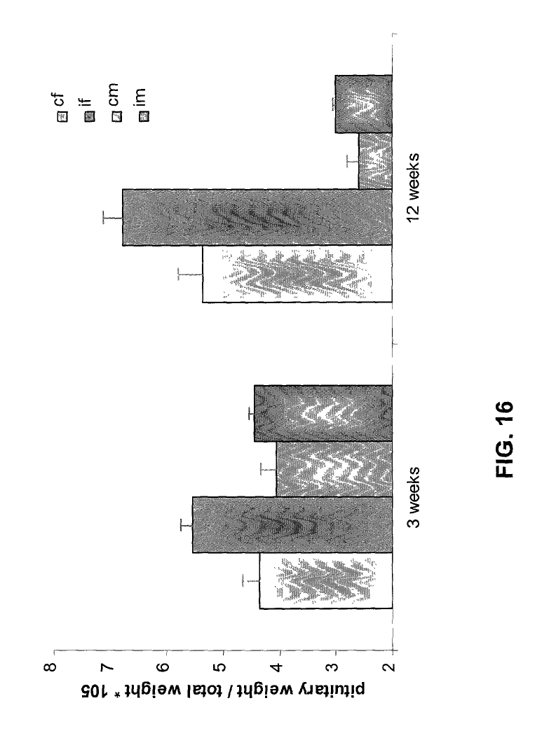

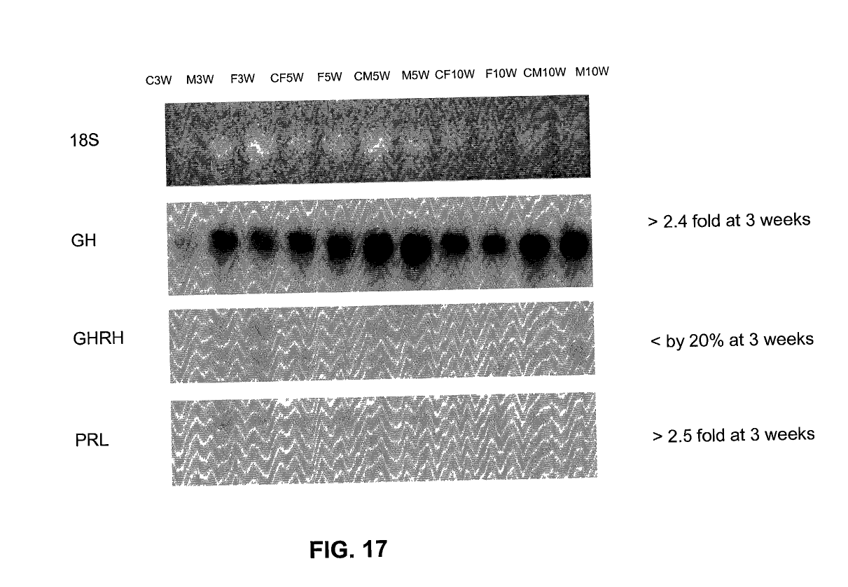

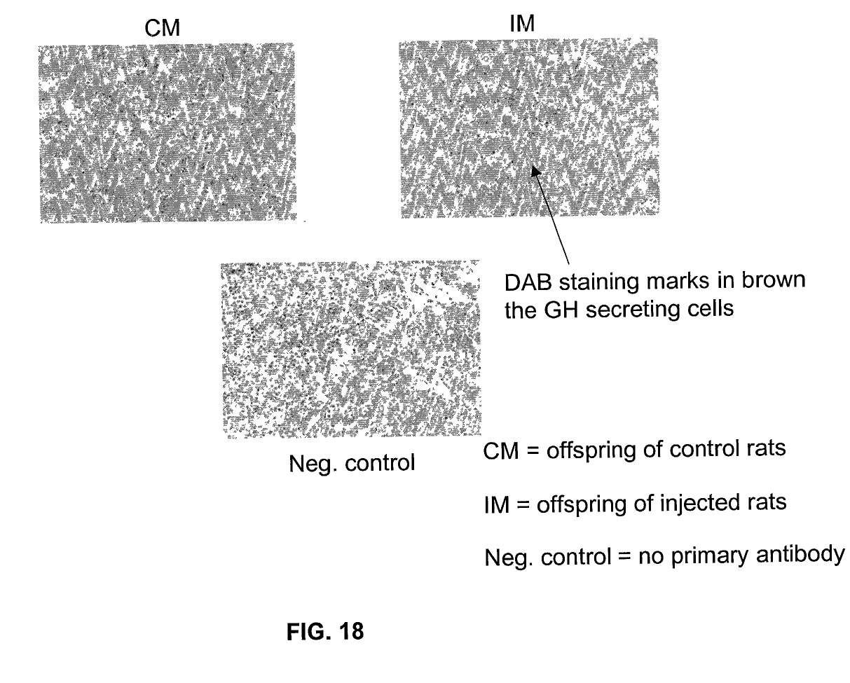

Pituitary glands were dissected within the first minutes post-mortem and weighed. The ratio of pituitary weight to total body weight was significantly increased up to 12 weeks after birth, predominantly in IF (FIG. 16). The increase in pituitary weight is most probably due to somatotrophs hyperplasia, as it is known that GHRH is capable of stimulating the synthesis and secretion of GH from the anterior pituitary and has a specific hypertrophic effect on somatotrophs (Morel et al., 1999; Murray et al., 2000). This is supported by hormonal (FIG. 17) and histological (FIG. 18) evidence. Northern blot analysis of pituitaries form injected animals showed a significant increase in the GH and PRL mRNA levels, combined with a diminution of the endogenous rat GHRH mRNA levels. With histology techniques, a specific anti-rat GH antibody illustrates the increase number of somatotrophs.

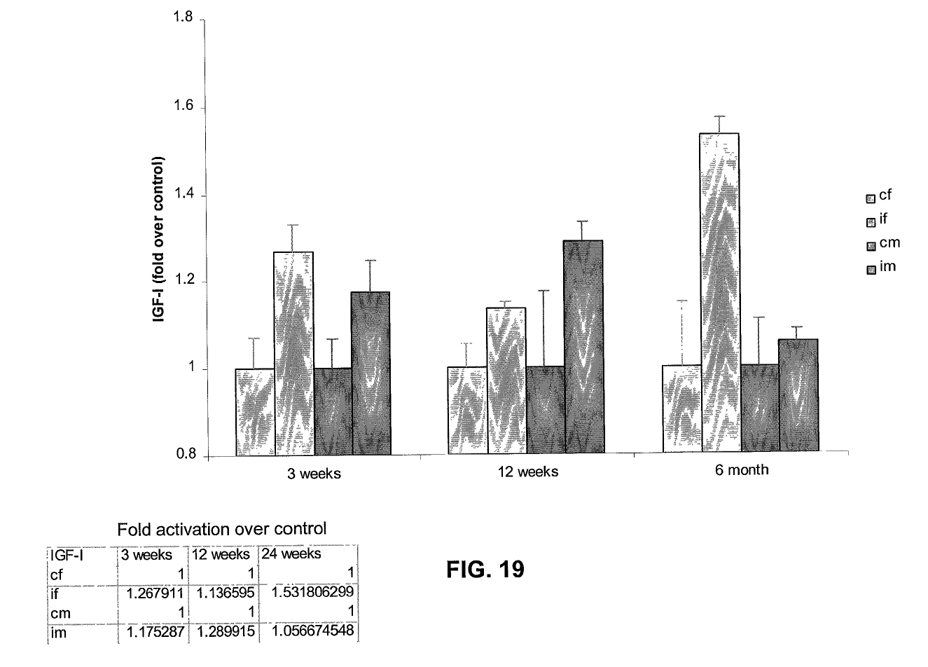

An indication of increased systemic levels of GHRH and GH is an increase in serum IGF-I concentration. Serum rat IGF-I was significantly higher in offspring of pSP-HV-GHRH injected rats to up to 24 weeks after birth, with p<0.05 at all time points tested (FIG. 19).

Organs (lungs, heart, liver, kidney, stomach, intestine, adrenals, gonads, brain) were collected and weighed. No associated pathology was observed in any of the animals. Among the nonviral techniques for gene transfer in vivo, the direct injection of plasmid DNA into muscle is simple, inexpensive, and safe, but applications of this methodology have been limited by the relatively low expression levels of the transferred DNA expression vectors. In a specific embodiment, in order to obtain regulation of growth and body composition by gene therapy it was necessary to utilize an innovative approach, wherein the target animals are not directly treated, but they have enhanced biological characteristics due to treatment of the pregnant mothers. Another significant improvement of the plasmid vector, such as the one described herein, was the employment of a gene that codes for a more stable GHRH analog, HV-GHRH (Draghia-Akli et al., 1999). Electrogene therapeutic transfer allows genes to be efficiently transferred and expressed in desired organs or tissues, and it is capable of providing long-term expression following a single administration. This method may represent a new approach for highly effective nucleic acid transfer that does not require viral genes or particles.

For large species such as pigs or cattle, the use of GHRH, the upstream stimulator of GH, is an alternate strategy that may increase not only growth performance or milk production, but more importantly, the efficiency of production from both practical and metabolic perspectives (Dubreuil et al., 1990). However, the high cost of the recombinant peptides and the required frequency of administration currently limit the widespread use of this treatment. These major drawbacks can be obviated by using a nucleic acid transfer approach to direct the ectopic production of GHRH, particularly when its production is sustained chronically.

Thus, enhanced animal growth occurred in offspring following a single electroporated injection of a plasmid expressing a mutated growth hormone releasing hormone (GHRH) cDNA, into the tibialis anterior muscles of adult pregnant rats. Newborn rats (F1) were significantly bigger at birth. Longitudinal weight and body composition studies showed a difference in between the two sexes with age. Hormonal and biochemical measurements were concordant with the growth pattern. F1 had larger pituitary glands, with somatotrophs hyperplasia and increased GH content. F1 plasma IGF-I levels were significantly elevated. In summary, these novel findings demonstrate that GHRH could be used to enhance certain animal characteristics throughout generations following plasmid-based gene therapy.

The following paragraphs describe the experiments performed in this Example.

DNA Constructs.

The plasmid pSPc5-12 contains a 360 bp SacI/BamHI fragment of the SPc5-12 synthetic promoter (Li et al., 1999) in the SacI/BamHI sites of pSK-GHRH backbone (Draghia-Akli et al., 1999). The mutated porcine GHRH cDNA were obtained by site-directed mutagenesis of human GHRH cDNA (Altered Sites II in vitro Mutagenesis System, Promega, Madison, Wis.). The mutated 228-bp fragment of porcine GHRH (part of exon 2, all exon 3 and part of exon 4), which encodes the 31 amino acid signal peptide and a mutated porcine GHRH (1-40)OH, is characterized by the following amino acid substitutions: Gly15 to Ala, Met27 to Leu and Ser28 to Asn, and conversion of Tyr1 to His, and Ala2 to Val. This fragment was cloned into the BamHI/Hind III sites of pSK-GHRH. hGH pA is a 3′ untranslated region and poly(A) signal from the human GH gene. Plasmids were grown in E. coli DH5α (Gibco BRL, Carlsbad, Calif.). Endotoxin-free plasmid (Qiagen Inc., Chatsworth, Calif., USA) preparations were diluted in PBS, pH 7.4 to 1 mg/ml.

Intramuscular Injection of Plasmid and Electroporation.

Time pregnant adult Wistar female rats were housed and cared for in the animal facility of Baylor College of Medicine, Houston, Tex. Animals were maintained under environmental conditions of 10 h light/14 h darkness, in accordance with NIH Guide, USDA and Animal Welfare Act guidelines, and the protocol was approved by the Institutional Animal Care and Use Committee. The experiment was repeated twice. On day 16 of gestation, the animals (n=20 group) were weighed and anesthetized using a combination of 42.8 mg/ml ketamine, 8.2 mg/ml xylazine and 0.7 mg/ml acepromazine, administered i.m. at a dose of 0.5-0.7 ml/kg. The left tibialis anterior muscle of rats was injected with 30 mg of pSP-HV-GHRH in 100 ml PBS using 0.3 cc insulin syringes (Becton-Dickinson, Franklin Lakes, N.J.). Control animals were injected with PBS. For both groups, the injection was followed by caliper electroporation, as described (Draghia-Akli et al., 1999). Briefly, two minutes after injection, the rat leg was placed in between a two needles electrode, 1 cm length, 26 gauge, 1 cm in between needles (Genetronics, San Diego, Calif.) and electric pulses were applied to the area. Three 60-ms pulses at a voltage of 100 V/cm were applied in one orientation, then the electric field was reversed, and three more pulses were applied in the opposite direction. The pulses were generated with a T-820 Electro Square Porator (Genetronics, San Diego, Calif.).

Offspring Studies.

All injected rats gave birth at 20-22 days of gestation. In the first study 240 offspring and in the second study 60 offspring were analyzed from birth to 5 month of age (birth, 2, 3, 6, 8, 12, 16, 22 weeks after birth). Body weights were recorded at these time points using the same calibrated balance. At the end of the experiment, body composition was performed post-mortem. Blood was collected, centrifuged immediately at 0° C., and stored at −80° C. prior to analysis. Organs (heart, liver, spleen, kidney, pituitary, brain, adrenals, skeletal muscles—tibialis anterior (TA), gastrocnemius (G), soleus (S), and extensor digitorum longus (EDL), carcass, fat from injected animals and controls were removed, weighed on an analytical balance and snap frozen in liquid nitrogen. Tibia length was measured and recorded.

Northern Blot Analysis of Pituitary.

Pituitaries were snap frozen and homogenized in solution D, and extracted. 20 mg of total RNA was DNase I treated, size separated in 1.5% agarose-formaldehyde gel and transferred to nylon membrane. The membranes were hybridized with a specific GHRH cDNA probe ³²P-labeled by random priming.

Rat IGF-I Radioimmunoassay.

Rat IGF-I was measured by specific radioimmunoassay (Diagnostic System Laboratories, Webster, Texas). The sensitivity of the assay was 0.8 ng/ml; intra-assay and inter-assay variation was 2.4% and 4.1% respectively.

Statistics.

Values shown in the figures are the mean ±s.e.m. Specific p values were obtained by comparison using Students t-test or ANOVA analysis. A p<0.05 was set as the level of statistical significance.

### REFERENCES CITED

### U.S. Patent Documents

U.S. Pat. No. 5,847,066 issued on Dec. 8, 1998 with Coy et al. listed as inventors.

U.S. Pat. No. 5,846,936 issued on Dec. 8, 1998 with Felix et al. listed as inventors.

U.S. Pat. No. 5,792,747 issued on Aug. 11, 1998 with Schally et al. listed as inventors.

U.S. Pat. No. 5,776,901 issued on Jul. 7, 1998 with Bowers et al. listed as inventors.

U.S. Pat. No. 5,756,264 issued on May 26, 1998 with Schwartz et al. listed as inventors.

U.S. Pat. No. 5,696,089 issued on Dec. 9, 1997 with Felix et al. listed as inventors.

U.S. Pat. No. 5,486,505 issued on Jan. 23, 1996 with Bowers et al. listed as inventors.

U.S. Pat. No. 5,292,721 issued on Mar. 8, 1994 with Boyd et al. listed as inventors.

U.S. Pat. No. 5,137,872 issued on Aug. 11, 1992 with Seely et al. listed as inventors.

U.S. Pat. No. 5,134,210 issued on Jul. 28, 1992 with Boyd et al. listed as inventors.

U.S. Pat. No. 5,084,442 issued on Jan. 28, 1992 with Felix et al. listed as inventors.

U.S. Pat. No. 5,061,690 issued on Oct. 29, 1991 with Kann et al. listed as inventors.

U.S. Pat. No. 5,036,045 issued on Jul. 30, 1991 with Thomer listed as the inventor.

U.S. Pat. No. 5,023,322 issued on Jun. 11, 1991 with Kovacs et al. listed as inventors.

U.S. Pat. No. 4,839,344 issued on Jun. 13, 1989 with Bowers et al. listed as inventors.

U.S. Pat. No. 4410,512 issued on Oct. 18, 1983 with Bowers et al. listed as inventors.

U.S. Pat. No. RE33,699 issued on Sep. 24, 1991 with Drengler listed as the inventor.

U.S. Pat. No. 4,833,166 issued on May 23, 1989 with Grosvenor et al. listed as inventors.

U.S. Pat. No. 4,228,158 issued on Oct. 14, 1980 with Momany et al. listed as inventors.

U.S. Pat. No. 4,228,156 issued on Oct. 14, 1980 with Momany et al. listed as inventors.

U.S. Pat. No. 4,226,857 issued on Oct. 7, 1980 with Momany et al. listed as inventors.

U.S. Pat. No. 4,224,316 issued on Sep. 23, 1980 with Momany e t al listed as inventors.

U.S. Pat. No. 4,223,021 issued on Sep. 16, 1980 with Momany et al . listed as inventors.

U.S. Pat. No. 4,223,020 issued on Sep. 16, 1980 with Momany et al . listed as inventors.

U.S. Pat. No. 4,223,019 issued on Sep. 16, 1980 with Momany et al. listed as inventors.

### Publications

Aihara, H. & Miyazaki, J. Nat. Biotechnol. 16, 867-870 (1998).

Albanese, A. and R. Stanhope. 1997. GH treatment induces sustained catch-up growth in children with intrauterine growth retardation: 7-year results. Horm. Res. 48:173-177.

Allen, D. B., A. C. Rundle, D. A. Graves, and S. L. Blethen. 1997. Risk of leukemia in children treated with human growth hormone: review and reanalysis. J. Pediatr. 131:S32-S36

Aramburo, C., Luna, M., Carranza, M., Reyes, M., Martinez-Coria, H., Scanes, C. G. (2000) Growth hormone size variants: changes in the pituitary during development of the chicken. Proc. Soc. Exp. Biol. Med. 223, 67-74

Argente, J., Pozo, J., Chowen, J. A. (1996) The growth hormone axis: control and effects. Hormone Research 45 Suppl 1, 9-11

Asa, S. L., Kovacs, K., Stefaneanu, L., Horvath, E., Billestrup, N., Gonzalez-Manchon, C., Vale, W. (1992) Pituitary adenomas in mice transgenic for growth hormone-releasing hormone. Endocrinology 131, 2083-2089

Azcona, C., A. Albanese, P. Bareille, and R. Stanhope. 1998. Growth hormone treatment in growth hormone-sufficient and -insufficient children with intrauterine growth retardation/Russell-Silver syndrome. Horm. Res. 50:22-27.

Barr, E., Leiden, J. M. (1991) Systemic delivery of recombinant proteins by genetically modified myoblasts. Science 254, 1507-1509

Bartke, A. 1998. Growth hormone and aging. Endocrine 8:103-108.

Benfield, M. R. and E. C. Kohaut. 1997. Growth hormone is safe in children after renal transplantation. J. Pediatr. 131:S28-S31

Bercu, B. B., R. F. Walker. 1997. Growth hormone secretagogues in children with altered growth. Acta Paediatrica 86:102-106.

Bergsma, D. J., Grichnik, S. M., Gossett, L. M. & Schwartz, R. J. Mol. Cell. Biol. 6, 2462-2475 (1986).

Bettan, M., Emmanuel, F., Darteil, R., Caillaud, J. M., Soubrier, F., Delaere, P., Branelec, D., Mahfoudi, A., Duverger, N., Scherman, D. (2000) High-level protein secretion into blood circulation after electric pulse-mediated gene transfer into skeletal muscle. Mol. Ther. 2, 204-210

Billestrup, N., Gonzalez-Manchon, C., Potter, E., Vale, W. (1990) Inhibition of somatotroph growth and growth hormone biosynthesis by activin in vitro. Mol. Endocrinol. 4, 356-362

Blethen, S. L. and A. C. Rundle. 1996. Slipped capital femoral epiphysis in children treated with growth hormone. A summary of the National Cooperative Growth Study experience. Horm. Res. 46:113-116.

Bowers, C. Y. 1998. Growth hormone-releasing peptide (GHRP). Cell Mol Life Sci. 54(12):1316-29.

Campbell, R. M., Y. Lee, J. Rivier, E. P. Heimer, A. M. Felix, and T. F. Mowles. 1991. GRF analogs and fragments: correlation between receptor binding, activity and structure. Peptides 12:569-574.

Chung, C. S., Etherton, T. D., Wiggins, J. P. (1985) Stimulation of swine growth by porcine growth hormone. J. Anim Sci. 60, 118-130

Corpas, E., S. M. Harman, and M. R. Blackman. 1993. Human growth hormone and human aging. Endocrine Reviews 14:20-39.

Corpas, E., S. M. Harman, M. A. Pineyro, R. Roberson, and M. R. Blackman. 1993. Continuous subcutaneous infusions of growth hormone (GH) releasing hormone 1-44 for 14 days increase GH and insulin-like growth factor-I levels in old men. Journal of Clinical Endocrinology & Metabolism 76:134-138.

Dahler, A., Wade, R. P., Muscat, G. E., Waters, M. J. (1994) Expression vectors encoding human growth hormone (hGH) controlled by human muscle-specific promoters: prospects for regulated production of hGH delivered by myoblast transfer or intravenous injection. Gene 145, 305-310

Davis, H. L., Whalen, R. G. & Demeneix, B. A. Hum. Gene Ther. 4, 151-159 (1993).

D'Costa, A. P., R. L. Ingram, J. E. Lenham, and W. E. Sonntag. 1993. The regulation and mechanisms of action of growth hormone and insulin-like growth factor 1 during normal aging. J. Reprod. Fert.—Supp. 46:87-98.

Dhawan, J., Pan, L. C., Pavlath, G. K., Travis, M. A., Lanctot, A. M., Blau, H. M. (1991) Systemic delivery of human growth hormone by injection of genetically engineered myoblasts. Science 254, 1509-1512

Dieguez C, Casanueva F F. 2000. Ghrelin: a step forward in the understanding of somatotroph cell function and growth regulation. Eur J Endocrinol. 142(5):413-7.

Draghia-Akli, R., Li, X. G., Schwartz, R. J., et al. Nat. Biotechnol. 15, 1285-1289 (1997).

Draghia-Akli, R. Fiorotto M L, Hill L A, Malone P B, Deaver D R, Schwartz R J Nat Biotechnol, 17(12), 1179-83 (1999).

Dubreuil, P., Petitclerc, D., Pelletier, G., Gaudreau, P., Farmer, C., Mowles, T F, Brazeau, P. (1990) Effect of dose and frequency of administration of a potent analog of human growth hormone-releasing factor on hormone secretion and growth in pigs. Journal of Animal Science 68, 1254-1268

Eicher, E. M. and W. G. Beamer. 1976. Inherited ateliotic dwarfism in mice. Characteristics of the mutation, little, on chromosome 6. J. Hered. 67:87-91.

Erling, A. 1999. Methodological considerations in the assessment of health-related quality of life in children. Acta Paediatrica Scandin.—Supp. 428:106-107. 0803-5326.

Esch, F. S., P. Bohlen, N. C. Ling, P. E. Brazeau, W. B. Wehrenberg, M. O. Thorner, M. J. Cronin, and R. Guillemin. 1982. Characterization of a 40 residue peptide from a human pancreatic tumor with growth hormone releasing activity. Biochemical & Biophysical Research Communications 109:152-158.

Etherton, T. D., Wiggins, J. P., Chung, C. S., Evock, C. M., Rebhun, J. F., Walton, P. E. (1986) Stimulation of pig growth performance by porcine growth hormone and growth hormone-releasing factor. Journal of Animal Science 63, 1389-1399

Etienne, M., Bonneau, M., Kann, G., Deletang, F. (1992) Effects of administration of growth hormone-releasing factor to sows during late gestation on growth hormone secretion, reproductive traits, and performance of progeny from birth to 100 kilograms live weight. Journal of Animal Science 70, 2212-2220

Everett, R. S., Gerrard, D. E., Grant, A. L. (2000) Factors affecting production of luciferase and epitope-tagged IGF-I in porcine muscle after DNA injection. J Endocrinol. 166, 255-263

Farmer, C., Petitclerc, D., Pelletier, G., Brazeau, P. (1992) Lactation performance of sows injected with growth hormone-releasing factor during gestation and(or) lactation.

Journal of Animal Science 70, 2636-2642

Faglia, G., Arosio, M., Bazzoni, N. (1992) Ectopic acromegaly. [Review]. Endocrinology & Metabolism Clinics of North America 21, 575-595

Frohman, M. A., T. R. Downs, P. Chomczynski, and L. A. Frohman. 1989. Cloning and characterization of mouse growth hormone-releasing hormone (GRH) complementary DNA: increased GRH messenger RNA levels in the growth hormone-deficient lit/lit mouse. Mol. Endocrinol. 3:1529-1536.

Frohman, L. A., J. L. Thominet, C. B. Webb, M. L. Vance, H. Uderman, J. Rivier, W. Vale, and M. O. Thorner . 1984. Metabolic clearance and plasma disappearance rates of human pancreatic tumor growth hormone releasing factor in man. J. Clin. Invest. 73:1304-1311.

Geffner, M. (1997) Effects of growth hormone and insulin-like growth factor I. Acta Paediatr. Suppl 423, 76-79

Gesundheit, N. and J. K. Alexander. 1995. Endocrine Therapy with Recombinant Hormones and Growth Factors. In Molecular Endocrinology: Basic Concepts and Clinical Correlations. B. D. Weintraub, editor. Raven Press, Ltd., New York. 491-507.

Gopinath, R., Etherton, T. D. (1989) Effects of porcine growth hormone on glucose metabolism of pigs: I. Acute and chronic effects on plasma glucose and insulin status. J. Anim Sci. 67, 682-688

Hataya, Y., Akamizu, T., Takaya, K., Kanamoto, N., Ariyasu, H., Saijo, M., Moriyama, K., Shimatsu, A., Kojima, M., Kangawa, K., Nakao, K. (2001) A low dose of ghrelin stimulates growth hormone (GH) release synergistically with GH-releasing hormone in humans. J. Clin. Endocrinol. Metab 86, 4552

Hattori, N., Saito, T., Yagyu, T., Jiang, B. H., Kitagawa, K., Inagaki, C. (2001) GH, GH receptor, GH secretagogue receptor, and ghrelin expression in human T cells, B cells, and neutrophils. J. Clin. Endocrinol. Metab 86, 4284-4291

Heptulla, R. A., S. D. Boulware, S. Caprio, D. Silver, R. S. Sherwin, and W. V. Tamborlane. 1997. Decreased insulin sensitivity and compensatory hyperinsulinemia after hormone treatment in children with short stature. J. Clin. Endocrinol. Metab. 82:3234-3238.

Horvath, T. L., Diano, S., Sotonyi, P., Heiman, M., Tschop, M. (2001) Minireview: ghrelin and the regulation of energy balance-a hypothalamic perspective . Endocrinology 142, 4163-4169

Howard, A. D., Feighner, S. D., Cully, D. F. Arena, J P., Liberator, P. A., Rosenblum, C. I., Hamelin, M., Hreniuk, D. L., Palyha, O. C., Anderson, J., Paress, P. S., Diaz, C., Chou, M., Liu, K. K., McKee, K. K., Pong, S. S., Chaung, L., Elbrecht, A., Dashkevicz, M., Heavens, R., Rigby, M., Sirinathsinghji, D., Dean, D. C., Melillo, D. G., Patchett, A. A., Nargund, R., Griffin, P. R., DeMartino, J. A., Gupta, S. K., Schaeffer, J. M., Smith R. G., Van der Ploeg, L. H. T. (1996) Receptor in pituitary and hypothalamus that functions growth hormone release. Science 273: 974-977.

Iranmanesh, A., G. Lizarralde, and J. D. Veldhuis. 1991. Age and relative adiposity are specific negative determinants of the frequency and amplitude of growth hormone (GH) secretory bursts and the half-life of endogenous GH in healthy men. Journal of Clinical Endocrinology & Metabolism 73:1081-1088.

Jacobs, P. A., P. R. Betts, A. E. Cockwell, J. A. Crolla, M. J. Mackenzie, D. O. Robinson, and S. A. Youings. 1990. A cytogenetic and molecular reappraisal of a series of patients with Turner's syndrome. Ann. Hum. Genet. 54:209-223.

Jaffe, H. A., C. Danel, G. Longenecker, M. Metzger, Y. Setoguchi, M. A. Rosenfeld, T. W. Gant, S. S. Thorgeirsson, L. D. Stratford-Perricaudet, M. Perricaudet, A. Pavirani, J. -P. Lecocq and R. G. Crystal. 1992. Adenovirus-mediated in vivo gene transfer and expression in normal rat liver. Nat Genet 1(5):372-8.

Key, L. L. J. and A. J. Gross. 1996. Response to growth hormone in children with chondrodysplasia. J. Pediatr. 128:S14-S17

Klindt, J., Yen, J. T., Buonomo, F. C., Roberts, A. J., Wise, T. (1998) Growth, body composition, and endocrine responses to chronic administration of insulin-like growth factor I and(or) porcine growth hormone in pigs. J. Anim Sci. 76, 2368-2381

Kojima M, Hosoda H, Date Y, Nakazato M, Matsuo H, Kangawa K. 1999. Ghrelin is a growth-hormone-releasing acylated peptide from stomach. Nature 402(6762):656-60.

Kubiak, T. M., C. R. Kelly, and F. Krabill. 1989. In vitro metabolic degradation of a bovine growth hormone-releasing factor analog Leu27-bGRF(1-29)NH2 in bovine and porcine plasma. Correlation with plasma dipeptidylpeptidase activity. Drug Metabolism & Disposition 17:393-397.

Li, X., Eastman, E. M., Schwartz, R. J., & Draghia-Akli, R. Nat. Biotechnol. 17. 3, 241-245 (1999).

Lopez-Calderon, A., Soto, L., Villanua, M. A., Vidarte, L., Martin, A. I. (1999) The effect of cyclosporine administration on growth hormone release and serum concentrations of insulin-like growth factor-I in male rats. Life Sci. 64, 1473-1483

Martin, R. A., D. L. Cleary, D. M. Guido, H. A. Zurcher-Neely, and T. M. Kubiak. 1993. Dipeptidyl peptidase IV (DPP-IV) from pig kidney cleaves analogs of bovine growth hormone-releasing factor (bGRF) modified at position 2 with Ser, Thr or Val. Extended DPP-IV substrate specificity? Biochimica et Biophysica Acta 1164:252-260.

Melmed, S. (1991) Extrapituitary Acromegaly. [Review]. Endocrinology & Metabolism Clinics of North America 20, 507-518

Miller, K. F., Bolt, D. J., Pursel, V. G., Hammer, R. E., Pinkert, C. A., Palmiter, R. D., Brinster, R. L. (1989) Expression of human or bovine growth hormone gene with a mouse metallothionein-1 promoter in transgenic swine alters the secretion of porcine growth hormone and insulin-like growth factor-I. J. Endocrinol. 120, 481-488

Mir, L. M., Bureau, M. F., Gehl, J., Rangara, R., Rouy, D., Caillaud, J. M., Delaere, P., Branellec, D., Schwartz, B., Scherman, D. (1999) High-efficiency gene transfer into skeletal muscle mediated by electric pulses. Proc. Natl. Acad. Sci. U.S.A 96, 4262-4267

Monti, L. D., P. Brambilla, A. Caumo, F. Magni, S. Omati, G. Nizzoli, B. di Natale, M. Galli-Kienle, C. Cobelli, G. Chiumello, and G. Pozza. 1997. Glucose turnover and insulin clearance after growth hormone treatment in girls with Turner's syndrome. Metabolism 46:1482-1488.

Morel, G., Gallego, R., Boulanger, L., Pintos, E., Garcia-Caballero, T., Gaudreau, P. (1999) Restricted presence of the growth hormone-releasing hormone receptor to somatotropes in rat and human pituitaries. Neuroendocrinology 70, 128-136

Muramatsu, T., Nakamura, A. & Park, H. M. Int. J. Mol. Med. 1, 55-62 (1998).

Murray, R. A., Maheshwari, H. G., Russell, E. J., Baumann, G. (2000) Pituitary hypoplasia in patients with a mutation in the growth hormone-releasing hormone receptor gene. AJNR Am. J Neuroradiol. 21, 685-689

Murray, R. D., Shalet, S. M. (2000) Growth hormone: current and future therapeutic applications. Expert. Opin. Pharmacother. 1, 975-990

Parks, J. S., R. W. Pfaffle, M. R. Brown, H. Abdul-Latif, and L. R. Meacham. 1995. Growth Hormone Deficiency. In Molecular Endocrinology: Basic Concepts and Clinical Correlations. B. D. Weintraub, editor. Raven Press, Ltd., New York. 473-490.

Pong, S. -S., Chaung, L. -Y. P., Dean, D. C., Nargund, R. P., Patchett, A. A. and Smith, R. G. (1996). Identification of a new G-protein-linked receptor for growth hormone secretagogues. Molecular Endocrinology 10: 57-61.

Pursel, V. G., Hammer, R. E., Bolt, D. J., Palmiter, R. D., Brinster, R. L. (1990) Integration, expression and germ-line transmission of growth-related genes in pigs. [Review] [33 refs]. Journal of Reproduction & Fertility—Supplement 41, 77-87

Pursel, V. G., Bolt, D. J., Miller, K. F., Pinkert, C. A., Hammer, R. E., Palmiter, R. D., Brinster, R. L. (1990) Expression and performance in transgenic pigs. J. Reprod. Fertil. Suppl 40:235-45, 235-245

Rawlings, S. R., Piuz, I., Schlegel, W., Bockaert, J., Journot, L. (1995) Differential expression of pituitary adenylate cyclase-activating polypeptide/vasoactive intestinal polypeptide receptor subtypes in clonal pituitary somatotrophs and gonadotrophs. Endocrinology 136, 2088-2098

Rosenbaum, P. L. and S. Saigal. 1996. Measuring health-related quality of life in pediatric populations: conceptual issues. In Quality of life and pharmacoeconomics in clinical trials. B. Spilker, editor. Lippincott-Raven Publishers, Philadelphia.

Rosenfeld, M. A., K Yoshimura, L. E. Stier, B. C. Trapnell, L. D. Stratford-Perricaudet, M. Perricaudet, W. Dalemans, S. Jallat, A. Mercenier, A. Pavirani, J. P. Lecocq, W. B. Guggino, R. G. Crystal. 1991. In vivo transfer of the human cystic fibrosis gene to the respiratory epithelium. Clinical Research 39 (2), 311A.

Rosenfeld, M. A., W Siegfried, K Yoshimura, K Yoneyama, M Fukayama, L E Stier, P K Paakko, P Gilardi, L D Stratford-Perricaudet, M Perricaudet, S. Jallat, A. Pavirani, J. -P. Lecocq, and R. G. Crystal. 1991. Adenovirus-mediated transfer of a recombinant alpha 1-antitrypsin gene to the lung epithelium in vivo. Science 252(5004):431-4.

Sano, T., Asa, S. L., Kovacs, K. (1988) Growth hormone-releasing hormone-producing tumors: clinical, biochemical, and morphological manifestations. Endocr. Rev. 9, 357-373

Savage, M. O., R. M. Beattie, C. Camacho-Hubner, J. A. Walker-Smith, and I. R. Sanderson. 1999. Growth in Crohn's disease. Acta Paediatrica Scandin—Supp. 428:89-92.

Scanlon, M. F., B. G. Issa, and C. Dieguez. 1996. Regulation of Growth Hormone Secretion. Hormone Research 46:149-154.

Shalet, S. M., B. M. Brennan, and R. E. Reddingius. 1997. Growth hormone therapy and malignancy. Horm. Res. 48 Suppl 4:29-32:29-32.

Skuse, D. H., K. Elgar, and E. Morris. 1999. Quality of life in Turner syndrome is related to chromosomal constitution: implication for genetic counseling and management. Acta Paediatrica Scandin.—Supp. 428:110-113.

Smith, V. G., Leman, A. D., Seaman, W. J., VanRavenswaay, F. (1991) Pig weaning weight and changes in hematology and blood chemistry of sows injected with recombinant porcine somatotropin during lactation. J. Anim Sci. 69, 3501-3510

Smith, R. G., Van der Ploeg, L. H. T., Cheng, K., Hickey, G. J., Wyvratt, Jr., M. J., Fisher, M. H., Nargund, R. P., Patchett, A. A. (1997) Peptidomimetic regulation of growth hormone (GH) secretion. Endocrine Reviews 18: 621-645.

Sohmiya, M., K. Ishikawa, and Y. Kato. 1998. Stimulation of erythropoietin secretion by continuous subcutaneous infusion of recombinant human GH in anemic patients with chronic renal failure. Eur. J. Endocrinol. 138:302-306.

Su, C. M., L. R. Jensen, E. P. Heimer, A. M. Felix, Y. C. Pan, and T. F. Mowles. 1991. In vitro stability of growth hormone releasing factor (GRF) analogs in porcine plasma. Hormone & Metabolic Research 23:15-21.

Tanaka, H., T. Kubo, T. Yamate, T. Ono, S. Kanzaki, and Y. Seino. 1998. Effect of growth hormone therapy in children with achondroplasia: growth pattern, hypothalamic-pituitary function, and genotype. Eur. J. Endocrinol. 138:275-280.

Tanner, J. W., Davis, S. K., McArthur, N. H., French, J. T. & Welsh, T. H., Jr. J. Endocrinol. 125, 109-115 (1990).

Thorner, M. O., Hartman, M. L., Vance, M. L., Pezzoli, S. S., Ampleford, E. J. (1995) Neuroendocrine regulation of growth hormone secretion. [Review]. Neuroscience & Biobehavioral Reviews 19, 465-468

Thorner, M. O., L. A. Frohman, D. A. Leong, J. Thominet, T. Downs, P. Hellmann, J. Chitwood, J. M. Vaughan, and W. Vale. 1984. Extrahypothalamic growth-hormone-releasing factor (GRF) secretion is a rare cause of acromegaly: plasma GRF levels in 177 acromegalic patients. Journal of Clinical Endocrinology & Metabolism 59:846-849.

Tomic, M., Zivadinovic, D., Van Goor, F., Yuan, D., Koshimizu, T., Stojilkovic, S. S. (1999) Expression of Ca(2+)-mobilizing endothelin(A) receptors and their role in the control of Ca(2+) influx and growth hormone secretion in pituitary somatotrophs. J Neurosci. 19, 7721-7731

Tripathy, S. K., Svensson, E. C., Black, H. B., et al. Proc. Natl. Acad. Sci. USA 93, 10876-10880 (1996).

Veldhuis, J. D. (1998) Neuroendocrine control of pulsatile growth hormone release in the human: relationship with gender. Growth Horm. IGF. Res. 8 Suppl B, 49-59

Walter, R., W. H. Simmons, and T. Yoshimoto. 1980. Proline specific endo- and exopeptidases. Mol. Cell Biochem. 30:111-127.

Watkins, S. L. 1996. Bone disease in patients receiving growth hormone. Kidney Int. Suppl. 53:S126-7:S126-S127

Wolff, J. A., Ludtke, J. J., Acsadi, G., Williams, P., Jani, A. (1992) Long-term persistence of plasmid DNA and foreign gene expression in mouse muscle. Human Molecular Genetics 1, 363-369

One skilled in the art readily appreciates that the patent invention is well adapted to carry out the objectives and obtain the ends and advantages mentioned as well as those inherent therein. Growth hormone, growth hormone releasing hormone, analogs, plasmids, vectors, pharmaceutical compositions, treatments, methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

What is claimed:

1. A method of improving or enhancing growth in an offspring from a female animal comprising the step of introducing an effective amount of a vector into cells of said female animal prior to or during gestation of said offspring, wherein said vector comprises a promoter; a nucleotide sequence; and a 3′ untranslated region, under conditions wherein the nucleotide sequence is expressed and wherein said introduction and expression of said vector results in improved or enhanced growth in said offspring.

2. The method of claim 1, wherein said cells of said female animal comprise diploid cells.

3. The method of claim 1, wherein said cells of said female animal comprise muscle cells.

4. The method of claim 1, wherein said nucleic acid sequence encodes a growth hormone releasing hormone or its analog.

5. The method of claim 4, wherein said growth hormone releasing hormone is SEQ ID NO:1, SEQ ID NO:8, or its respective analog.

6. The method of claim 1, wherein said promoter comprises a synthetic myogenic promoter.

7. The method of claim 1, wherein said 3′ untranslated region comprises a hGH 3′ untranslated region.

8. The method of claim 1, wherein said vector is introduced into said cells of said female animal by electroporation, through a viral vector, in conjunction with a carrier, by parenteral route, or a combination thereof.

9. The method of claim 1, wherein said female animal is a human, a pet animal, a farm animal, a food animal, or a work animal.

10. The method of claim 1, wherein said female animal is a human, pig, cow, sheep, goat or chicken.

11. The vector of claim 1, wherein said vector is a plasmid, a viral vector, a liposome, a cationic lipid, or a combination thereof.

12. The method of claim 1, wherein said vector is introduced into said female in a single administration.

13. The method of claim 1, wherein said introduction occurs during the third trimester of gestation of said offspring.

14. The method of claim 1, further comprising the step of administering to said female a ligand for a growth hormone secretagogue receptor.

15. The method of claim 14, wherein said ligand administration is oral.

16. A method of increasing levels of growth hormone in an offspring from a female animal comprising the step of introducing an effective amount of a vector into cells of said female animal prior to or during gestation of said offspring, wherein said vector comprises a promoter; a nucleotide sequence; and a 3′ untranslated region, under conditions wherein the nucleotide sequence is expressed and wherein said introduction and expression of said vector results in increased levels of growth hormone in said offspring.

17. The method of claim 16, wherein said cells of said female animal comprise diploid cells.

18. The method of claim 16, wherein said cells of said female animal comprise muscle cells.

19. The method of claim 16, wherein said nucleic acid sequence encodes a growth hormone releasing hormone or its analog.

20. The method of claim 19, wherein said growth hormone releasing hormone is SEQ ID NO:1, SEQ ID NO:8, or its respective analog.

21. The method of claim 16, wherein said promoter comprises a synthetic myogenic promoter.

22. The method of claim 16, wherein said 3′ untranslated region comprises a hGH 3′ untranslated region.

23. The method of claim 16, wherein said vector is introduced into said cells of said female animal by electroporation, through a viral vector, in conjunction with a carrier, by parenteral route, or a combination thereof.

24. The method of claim 16, wherein said female animal is a human, a pet animal, a farm animal, a food animal, or a work animal.

25. The method of claim 16, wherein said female animal is a human, pig, cow, sheep, goat or chicken.

26. The vector of claim 16, wherein said vector is a plasmid, a viral vector, a liposome, a cationic lipid, or a combination thereof.

27. The method of claim 16, wherein said vector is introduced into said female in a single administration.

28. The method of claim 16, wherein said introduction occurs during the third trimester of gestation of said offspring.

29. The method of claim 16, further comprising the step of administering to said female a ligand for a growth hormone secretagogue receptor.

30. The method of claim 29, wherein said ligand administration is oral.

31. A method of increasing lean body mass in an offspring from a female animal comprising the step of introducing an effective amount of a vector into cells of said female animal prior to or during gestation of said offspring, wherein said vector comprises a promoter; a nucleotide sequence; and a 3′ untranslated region, under conditions wherein the nucleotide sequence is expressed and wherein said introduction and expression of said vector results in increased lean body mass in said offspring.

32. The method of claim 31, wherein said cells of said female animal comprise diploid cells.

33. The method of claim 31, wherein said cells of said female animal comprise muscle cells.

34. The method of claim 31, wherein said nucleic acid sequence encodes a growth hormone releasing hormone or its analog.

35. The method of claim 34, wherein said growth hormone releasing hormone is SEQ ID NO:1, SEQ ID NO:8, or its respective analog.

36. The method of claim 31, wherein said promoter comprises a synthetic myogenic promoter.

37. The method of claim 31, wherein said 3′ untranslated region comprises a hGH 3′ untranslated region.

38. The method of claim 31, wherein said vector is introduced into said cells of said female animal by electroporation, through a viral vector, in conjunction with a carrier, by parenteral route, or a combination thereof.

39. The method of claim 31, wherein said female animal is a human, a pet animal, a farm animal, a food animal, or a work animal.

40. The method of claim 31, wherein said female animal is a human, pig, cow, sheep, goat or chicken.

41. The vector of claim 31, wherein said vector is selected from the group consisting of a plasmid, a viral vector, a liposome, a cationic lipid, or a combination thereof.

42. The method of claim 31, wherein said vector is introduced into said female in a single administration.

43. The method of claim 31, wherein said introduction occurs during the third trimester of gestation of said offspring.

44. The method of claim 31, further comprising the step of administering to said female a ligand for a growth hormone secretagogue receptor.

45. The method of claim 44, wherein said ligand administration is oral.

46. A method of increasing levels of IGF-I in an offspring from a female animal comprising the step of introducing an effective amount of a vector into cells of said female animal prior to or during gestation of said offspring, wherein said vector comprises a promoter; a nucleotide sequence; and a 3′ untranslated region, under conditions wherein the nucleotide sequence is expressed and wherein said introduction and expression of said vector results in increased levels of IGF-I in said offspring.

47. The method of claim 46, wherein said cells of said female animal comprise diploid cells.

48. The method of claim 46, wherein said cells of said female animal comprise muscle cells.

49. The method of claim 46, wherein said nucleic acid sequence encodes a growth hormone releasing hormone or its analog.

50. The method of claim 49, wherein said growth hormone releasing hormone is SEQ ID NO:1, SEQ ID NO:8, or its respective analog.

51. The method of claim 46, wherein said promoter comprises a synthetic myogenic promoter.

52. The method of claim 46, wherein said 3′ untranslated region comprises a hGH 3′ untranslated region.

53. The method of claim 46, wherein said vector is introduced into said cells of said female animal by electroporation, through a viral vector, in conjunction with a carrier, by parenteral route, or a combination thereof.

54. The method of claim 46, wherein said female animal is a human, a pet animal, a farm animal, a food animal, or a work animal.

55. The method of claim 46, wherein said female animal is a human, pig, cow, sheep, goat or chicken.

56. The vector of claim 46, wherein said vector is a plasmid, a viral vector, a liposome, a cationic lipid, or a combination thereof.

57. The method of claim 46, wherein said vector is introduced into said female in a single administration.

58. The method of claim 46, wherein said introduction occurs during the third trimester of gestation of said offspring.

59. The method of claim 46, further comprising the step of administering to said female a ligand for a growth hormone secretagogue receptor.

60. The method of claim 59, wherein said ligand administration is oral.

61. A method of increasing feed efficiency in an offspring from a female animal comprising the step of introducing an effective amount of a vector into cells of said female animal prior to or during gestation of said offspring, wherein said vector comprises a promoter; a nucleotide sequence; and a 3′ untranslated region, under conditions wherein the nucleotide sequence is expressed and wherein said introduction and expression of said vector results in increased feed efficiency in said offspring.

62. The method of claim 61, wherein said cells of said female animal comprise diploid cells.

63. The method of claim 61, wherein said cells of said female animal comprise muscle cells.

64. The method of claim 61, wherein said nucleic acid sequence encodes a growth hormone releasing hormone or its analog.

65. The method of claim 64, wherein said growth hormone releasing hormone is SEQ ID NO:1, SEQ ID NO:8, or its respective analog.

66. The method of claim 61, wherein said promoter comprises a synthetic myogenic promoter.

67. The method of claim 61, wherein said 3′ untranslated region comprises a hGH 3′ untranslated region.

68. The method of claim 61, wherein said vector is introduced into said cells of said female animal by electroporation, through a viral vector, in conjunction with a carrier, by parenteral route, or a combanation thereof.

69. The method of claim 61, wherein said female animal is a human, a pet animal, a farm animal, a food animal, or a work animal.

70. The method of claim 61, wherein said female animal is a human, pig, cow, sheep, goat and chicken.

71. The vector of claim 61, wherein said vector is a plasmid, a viral vector, a liposome, a cationic lipid, or a combination thereof.

72. The method of claim 61, wherein said vector is introduced into said female in a single administration.

73. The method of claim 61, wherein said introduction occurs during the third trimester of gestation of said offspring.

74. The method of claim 61, further comprising the step of administering to said female a ligand for a growth hormone secretagogue receptor.

75. The method of claim 74, wherein said ligand administration is oral.

76. A method of increasing the rate of growth in an offspring from a female animal comprising the step of introducing an effective amount of a vector into cells of said female animal prior to or during gestation of said offspring, wherein said vector comprises a promoter; a nucleotide sequence; and a 3′ untranslated region, under conditions wherein the nucleotide sequence is expressed and wherein said introduction and expression of said vector results in increased rate of growth in said offspring.

77. The method of claim 76, wherein said cells of said female animal comprise diploid cells.

78. The method of claim 76, wherein said cells of said female animal comprise muscle cells.

79. The method of claim 76, wherein said nucleic acid sequence encodes a growth hormone releasing hormone or its analog.

80. The method of claim 79, wherein said growth hormone releasing hormone is SEQ ID NO:1, SEQ ID NO:8, or its respective analog.

81. The method of claim 76, wherein said promoter comprises a synthetic myogenic promoter.

82. The method of claim 76, wherein said 3′ untranslated region comprises a hGH 3′ untranslated region.

83. The method of claim 76, wherein said vector is introduced into said cells of said female animal by electroporation, through a viral vector, in conjunction with a carrier, by parenteral route, or a combination thereof.

84. The method of claim 76, wherein said female animal is a human, a pet animal, a farm animal, a food animal, or a work animal.

85. The method of claim 76, wherein said female animal is a human, pig, cow, sheep, goat or chicken.

86. The vector of claim 76, wherein said vector is a plasmid, a viral vector, a liposome, a cationic lipid, or a combination thereof.

87. The method of claim 76, wherein said vector is introduced into said female in a single administration.

88. The method of claim 76, wherein said introduction occurs during the third trimester of gestation of said offspring.

89. The method of claim 76, further comprising the step of administering to said female a ligand for a growth hormone secretagogue receptor.

90. The method of claim 89, wherein said ligand administration is oral.

91. A method of increasing the ratio of somatotrophs to other hormone-producing cells in a pituitary gland of an offspring from a female animal comprising the step of introducing an effective amount of a vector into cells of said female animal prior to or during gestation of said offspring, wherein said vector comprises a promoter; a nucleotide sequence; and a 3′ untranslated region, under conditions wherein the nucleotide sequence is expressed and wherein said introduction and expression of said vector results in ratio of somatotrophs to other hormone-producing cells in a pituitary gland in said offspring.

92. The method of claim 91, wherein said cells of said female animal comprise diploid cells.

93. The method of claim 91, wherein said cells of said female animal comprise muscle cells.

94. The method of claim 91, wherein said nucleic acid sequence encodes a growth hormone releasing hormone or its analog.

95. The method of claim 94, wherein said growth hormone releasing hormone is SEQ ID NO:1, SEQ ID NO:8, or its respective analog.

96. The method of claim 91, wherein said promoter comprises a synthetic myogenic promoter.

97. The method of claim 91, wherein said 3′ untranslated region comprises a hGH 3′ untranslated region.

98. The method of claim 91, wherein said vector is introduced into said cells of said female animal by electroporation, through a viral vector, in conjunction with a carrier, by parenteral route, or a combination thereof.

99. The method of claim 91, wherein said female animal is a human, a pet animal, a farm animal, a food animal, or a work animal.

100. The method of claim 91, wherein said female is an animal selected from the group consisting of human, pig, cow, sheep, goat and chicken.

101. The vector of claim 91, wherein said vector is a plasmid, a viral vector, a liposome, a cationic lipid, or a combination thereof.

102. The method of claim 91, wherein said vector is introduced into said female in a single administration.

103. The method of claim 91, wherein said introduction occurs during the third trimester of gestation of said offspring.

104. The method of claim 91, further comprising the step of administering to said female a ligand for a growth hormone secretagogue receptor.

105. The method of claim 104, wherein said ligand administration is oral.

106. The method of claim 91, wherein said hormone-producing cells are selected from the group consisting of corticotrophs, lactotrophs and gonadotrophs.

107. A method for delaying birth of an offspring from a female animal comprising the step of introducing an effective amount of a vector into cells of said female animal prior to or during gestation of said offspring, wherein said vector comprises a promoter; a nucleotide sequence; and a 3′ untranslated region, under conditions wherein the nucleotide sequence is expressed and wherein said introduction and expression of said vector results in delayed birth in said offspring.

108. The method of claim 107, wherein said cells of said female animal comprise diploid cells.

109. The method of claim 107, wherein said cells of said female animal comprise muscle cells.

110. The method of claim 107, wherein said nucleic acid sequence encodes a growth hormone releasing hormone or its analog.

111. The method of claim 110, wherein said growth hormone releasing hormone is SEQ ID NO:1, SEQ ID NO:8, or its respective analog.

112. The method of claim 107, wherein said promoter comprises a synthetic myogenic promoter.

113. The method of claim 107, wherein said 3′ untranslated region comprises a hGH 3′ untranslated region.

114. The method of claim 107, wherein said vector is introduced into said cells of said female animal by electroporation, through a viral vector, in conjunction with a carrier, by parenteral route, or a combination thereof.

115. The method of claim 107, wherein said female animal is a human, a pet animal, a farm animal, a food animal, or a work animal.

116. The method of claim 107, wherein said female animal is a human, pig, cow, sheep, goat or chicken.

117. The vector of claim 107, wherein said vector is a plasmid, a viral vector, a liposome, a cationic lipid, or a combination thereof.

118. The method of claim 107, wherein said vector is introduced into said female in a single administration.

119. The method of claim 107, wherein said introduction occurs during the third trimester of gestation of said offspring.

120. The method of claim 107, further comprising the step of administering to said female a ligand for a growth hormone secretagogue receptor.

121. The method of claim 120, wherein said ligand administration is oral.

122. A method of increasing milk production in an animal comprising the step of introducing an effective amount of a vector into cells of said animal, wherein said vector comprises a promoter; a nucleotide sequence; and a 3′ untranslated region, under conditions wherein the nucleotide sequence is expressed and wherein said introduction and expression of said vector results in increased milk production in said animal.

123. The method of claim 122, wherein said cells of said animal comprise diploid cells.

124. The method of claim 122, wherein said cells of said animal comprise muscle cells.

125. The method of claim 122, wherein said nucleic acid sequence encodes a growth hormone releasing hormone or its analog.

126. The method of claim 125, wherein said growth hormone releasing hormone is SEQ ID NO:1, SEQ ID NO:8, or its respective analog.

127. The method of claim 122, wherein said promoter comprises a synthetic myogenic promoter.

128. The method of claim 122, wherein said 3′ untranslated region comprises a hGH 3′ untranslated region.

129. The method of claim 122, wherein said vector is introduced into said cells of said female animal by electroporation, through a viral vector, in conjunction with a carrier, by parenteral route, or a combination thereof.

130. The method of claim 122, wherein said female animal is a human, a pet animal, a farm animal, a food animal, or a work animal.

131. The method of claim 122, wherein said animal is a human, pig, cow, sheep, goat or chicken.

132. The vector of claim 122, wherein said vector is a plasmid, a viral vector, a liposome, a cationic lipid, or a combination thereof.

133. The method of claim 122, wherein said vector is introduced into said animal in a single administration.

134. The method of claim 122, wherein said introduction occurs during the third trimester of gestation of said offspring.

135. The method of claim 122, further comprising the step of administering to said female a ligand for a growth hormone secretagogue receptor.

136. The method of claim 135, wherein said ligand administration is oral.
